(19) United States
(12) Patent Application Publication
(10) Pub. No.: US 2012/0015344 A1
(43) Pub. Date: Jan. 19, 2012

(54) Methods, compositions, and apparatus for the detection of viral strains

(75) Inventors: Evangelyn C. Alocilia, East Lansing, MI (US); Tracy L. Kamikawa, Centreville, VA (US); Dorothy Elizabeth Scott, Rockville, MD (US); Michael C. Kennedy, (US); Pei Zhang, (US); Malgorzata Mikolajczyk, Boonsboro, MD (US); Lilin Zhong, (US)

(73) Assignee: 1. Board of Trustees of MICHIGAN STATE UNIVERSITY, East Lansing, MI (US)

(21) Appl. No.: 13/068,427

(22) Filed: May 11, 2011

Related U.S. Application Data
(60) Provisional application No. 61/334,930, filed on May 14, 2010.

(51) Int. Cl. C12Q 1/70; C08G 73/02; C12M 1/34; C12N 7/00; B82Y 15/00

(52) U.S. Cl. 435/5; 435/287.2; 435/235.1; 525/54.1; 977/773; 977/902

(57) ABSTRACT

The disclosure generally relates to a particulate composition formed from a conductive polymer bound to magnetic nanoparticles. The particulate composition can be formed into a biologically enhanced, electrically active magnetic (BEAM) nanoparticle composition by further including a binding pair member (e.g., an antibody or a fragment thereof that specifically recognizes a virus strain or a virus surface protein) bound to the conductive polymer of the particulate composition. The disclosure further provides compositions, kits, detection apparatus, and methods for detecting specific viral strains including those with pandemic potential. In the various embodiments, a triplex including the BEAM nanoparticle, a virus or virally derived material (e.g. strain- and/or strain subtype specific viral surface protein or fragments thereof), and a viral strain subtype-specific binding pair member (e.g., a glycan that recognizes a specific virus strain subtype) is formed and detected, such as by use of a biosensor.

### CROSS-REFERENCE TO RELATED APPLICATION

Priority is claimed to U.S. Provisional Application No. 61/334,930, filed May 14, 2010, the disclosure of which is incorporated herein by reference in its entirety.

### STATEMENT OF GOVERNMENT INTEREST

This invention was made with government support from the National Science Foundation under grant number NSFDGE-0237003. The government has certain rights in the invention.

### BACKGROUND OF THE DISCLOSURE

1. Field of the Disclosure

The disclosure generally relates to a biologically enhanced, electrically active magnetic (BEAM) nanoparticle composition formed from a conductive polymer (e.g., conductive polyanilines, polypyrroles, polythiophenes) bound to magnetic nanoparticles (e.g., Fe(II)— and/or Fe(III)-based ferromagnetic magnetic metal oxides) and further including a binding pair member (e.g., an antibody or a fragment thereof that specifically recognizes a virus strain or a virus surface protein) bound to the conductive polymer. In particular embodiments, the disclosure provides compositions, kits, detection apparatus, and methods for detecting specific viral strains including those with pandemic potential. In the various embodiments, a triplex including the BEAM nanoparticle, a virus or virally derived material (e.g. strain- and/or strain subtype specific viral surface protein or fragments thereof), and a viral strain subtype-specific binding pair member (e.g., a glycan that recognizes a specific virus strain subtype) is formed, and the formation of said triplex is detected (e.g., by use of a biosensor).

2. Brief Description of Related Technology

The recent swine-origin H1N1 pandemic has brought attention to Influenza A virus (FLUAV), a causative agent of influenza infection. FLUAV has long been the cause of worldwide pandemics and, more commonly, annual epidemics, and may be found in a range of virulences and strains. FLUAV is an acute viral disease agent targeting the respiratory tract, and is a genus of the Orthomyxoviridae family (Werner and Harder, 2006). Globally, FLUAV annually affects millions of people and also impacts various animal species. The surface glycoproteins hemagglutinin (HA) and neuraminidase (NA) characterize each strain by serotype (Stevens et al., 2006). HA dictates host specificity and host cell entry and determines the extent of host infection (Stevens et al., 2006; Wiley and Skehel, 1987). Of the sixteen known HA and nine NA serotypes, all circulate in the avian population, in particular the Orders Anseriformes and Charadriiformes, leading to the common belief that birds act as the main FLUAV reservoir (Stevens et al., 2006; Webster et al., 1992). These hosts typically maintain FLUAV asymptomatically (Ellis et al., 2004; Sturm-Ramirez et al., 2004). However, highly pathogenic H5N1 has led to death and depopulation in poultry and wild waterfowl populations, and has presented a continuous threat throughout Asia, Europe, and Africa from 2003 to the present, with sporadic related infections in humans with close contact to infected species (Magalhaes et al., 2010; Neumann et al., 2010). Only three HA and two NA have adapted sufficiently to become human-transmissible, namely H1N1, H2N2, H3N2, and H1N2 (Neumann et al., 2010).

In the U.S., influenza viruses cause 36,000 deaths and 200,000 hospitalizations annually with costs of $10 billion, in association with seasonal epidemics resulting from antigenic drift (HSC, 2005). This drift causes minor changes in HA due to antigenic pressure from the prominent circulating HA type (Wright and Webster, 2001). Additionally, antigenic shifting may lead to a global FLUAV outbreak, or pandemic, with high mortality rates (Gurtler, 2006; Wright and Webster, 2001). Of the three historical pandemic strains, H1N1, H2N2, and H3N2, the 1918-1919 H1N1 “Spanish flu” was the most virulent, resulting in 20-40 million deaths (Reid et al., 2001; Stevens et al., 2006). Antigenic shifts cause a replacement of the genomic RNA segment encoding HA, allowing FLUAV to rapidly adapt from one animal species to another. The 2009 H1N1 pandemic originated from a swine FLUAV that has been circulating in pig herds for decades, and demonstrates how quickly a FLUAV that gains human-to-human transmissibility can spread worldwide (Michaelis et al., 2009).

Transmission of FLUAV infections, and thus pandemic potential, is dependent upon FLUAV HA receptor specificity for host glycan receptors. Avian FLUAV preferentially bind glycans with terminal sialic acids connected to galactose by α2,3 linkages in the lower respiratory tract, whereas human FLUAV preferentially bind to α2,6-linked sialic acids in the nose and throat. Avian FLUAV are of concern to human health due to the widely held belief that a highly pathogenic avian FLUAV could achieve human infectivity and transmissibility, and thus human pandemic potential, due to antigenic shifting (Blixt et al., 2004; Stevens et al., 2006).

Influenza virus is typically analyzed by well established conventional virological methods (Amano and Cheng, 2005). Viral isolation culture with immunocytological confirmation remains the “gold standard” for virus detection. Other methods include complement fixation (CF), hemagglutinin-inhibition (HI), and PCR. All require hours to several days for culture and detection. Influenza antigens or enzymes may also be directly detected by commercial diagnostic test kits, which typically detect viral antigen using anti-influenza antibodies. Few differentiate between influenza A and B. These kits require 30 minutes for testing with sensitivity from 10-70% (Amano and Cheng, 2005; Faix et al., 2009). These methods reveal a need for rapid technology which offers quantitative results as opposed to subjective color change assessments.

Detection technologies employing magnetic particles or microbeads have been used. These particles bind with the target analyte in a sample being tested, for example using a binding pair member specific to the target analyte, and are then typically isolated or separated out of solution magnetically. Once isolation has occurred, other testing may be conducted to detect the presence of analyte-bound particles. For example, various types of immunoassays based upon detecting a signal from a capture reagent are described in U.S. Pat. No. 5,620,845 to Gould et al.; U.S. Pat. No. 4,486,530 to David et al.; U.S. Pat. No. 5,559,041 to Kang et al.; U.S. Pat. No. 5,656,448 to Kang et al.; U.S. Pat. No. 5,728,587 to Kang et al.; U.S. Pat. No. 5,695,928 to Stewart et al.; U.S. Pat. No. 5,169,789 to Bernstein et al.; U.S. Pat. Nos. 5,177,014, 5,219,725, and 5,627,026 to O'Conner et al.; U.S. Pat. No. 5,976,896 to Kumar et al.; U.S. Pat. Nos. 4,939,096 and 4,965,187 to Tonelli; U.S. Pat. No. 5,256,372 to Brooks et al.; U.S. Pat. Nos. 5,166,078 and 5,356,785 to McMahon et al.; U.S. Pat. Nos. 5,726,010, 5,726,013, and 5,750,333 to Clark; U.S. Pat. Nos. 5,518,892, 5,753,456, and 5,620,895 to Naqui et al.; U.S. Pat. Nos. 5,700,655 and 5,985,594 to Croteau et al.; and U.S. Pat. No. 4,786,589 to Rounds et al. The aforementioned U.S. patents are hereby incorporated herein by reference herein in their entireties.

Alocilja et al. U.S. Publication Nos. 2003/0153094, 2008/0314766, 2009/0123939, generally relate to biosensor devices and/or BEAM nanoparticle compositions and are incorporated herein by reference in their entireties.

### SUMMARY

In one aspect, the disclosure relates to a biologically enhanced, electrically active magnetic (BEAM) nanoparticle composition comprising: (a) a particulate composition comprising a conductive polymer bound to magnetic nanoparticles; and (b) a binding pair member bound to the conductive polymer of the particulate composition, wherein the binding pair member is an antibody or a fragment thereof that specifically recognizes a virus strain or a virus surface protein. In an embodiment, the binding pair member specifically recognizes a hemagglutinin serotype as the virus strain. In another embodiment, the binding pair member specifically recognizes and is capable of binding a hemagglutinin as the virus surface protein (e.g., the binding pair member is an antibody that specifically recognizes an influenza B hemagglutinin virus surface protein selected from the group consisting of H1, H2, H3, and H5). In a particular embodiment of the BEAM nanoparticle composition, (i) the magnetic nanoparticles comprise at least one of Fe(II) and Fe(III); and, (ii) the conductive polymer is selected from the group consisting of polyanilines, polypyrroles, polythiophenes, derivatives thereof, combinations thereof, blends thereof with other polymers, and copolymers of the monomers thereof.

In another aspect, the disclosure relates to a kit comprising: (a) the biologically enhanced, electrically active magnetic (BEAM) nanoparticle composition according to any of its various embodiments, and (b) a further binding pair member that specifically recognizes a subtype of the virus strain or the virus surface protein specifically recognized by the binding pair member of the BEAM nanoparticle composition. In an embodiment, the subtype has receptor specificity for a host cell glycan receptor with terminal sialic acids dependent upon the linkage of the sialic acid to a saccharide moiety on the receptor, and the receptor specificity can confer at least one of human infectivity and human to human transmissibility to the virus strain. Suitably, the binding pair member and the further binding pair member are capable of simultaneously or sequentially binding a virus strain or a virus surface protein, thereby forming a triplex comprising the binding pair member of the BEAM nanoparticle and the further binding pair member bound to the virus strain or said virus surface protein.

Various refinements of the kit are possible. For example, the further binding pair member can be a glycan that preferentially binds host cell glycan receptors, the glycan comprising α2,6-, α2,3-, or α2,8-linked sialic acid and optionally further comprising a conjugating moiety selected from the group consisting of avidin, biotin, and streptavidin. The further binding pair member can be a glycan that preferentially binds host cell glycan receptors, the glycan comprising α2,6-linked sialic acid. In an embodiment, the further binding pair member is a glycan selected from the group consisting of: Neu5Acα2-3Galβ1-4[Fucα1-3]GlcNAcβ-SpNH-LC-LC-Biotin; Neu5Acα2-3Galβ1-4GlcNAcβ-SpNH-LC-LC-Biotin; Neu5Acα2-8Neu5Acα2-8Neu5Acα2-3Galβ1-4Glcβ-SpNH-LC-LC-Biotin; Neu5Acα2-6Galβ1-4GlcNAcβ-SpNH-LC-LC-Biotin; Neu5Acα2-3Galβ1-4[Fucα1-3]GlcNAcβ-SpNH-LC-LC; Neu5Acα2-3Galβ1-4GlcNAcβ-SpNH-LC-LC; Neu5Acα2-8Neu5Acα2-8Neu5Acα2-3Galβ1-4Glcβ-SpNH-LC; Neu5Acα2-6Galβ1-4GlcNAcβ-SpNH-LC-LC; Neu5Acα2-3Galβ1-4[Fucα1-3]GlcNAcβ-Osp-LC-LC; Neu5Acα2-3Galβ1-4[Fucα1-3]GlcNAcβ-Osp-LC-LC-Biotin; Neu5Acα2-3[Galβ1-4GlcNAcβ1-3]₂β-SpNH-LC-LC-Biotin; Neu5Acα2-6[Galβ1-4GlcNAcβ1-3]₂βSpNH-LC-LC-Biotin; Neu5Acα2-3[GalNAcβ1-4]Galβ1-4GlcNAcβ-SpNH-LC-LC-Biotin; Neu5Acα2-3Galβ1-4[Fucα1-3]GlcNAcβ-Osp-LC-LC; Neu5Acα2-3[Galβ1-4GlcNAcβ1-3]₂β-SpNH-LC-LC; Neu5Acα2-6[Galβ1-4GlcNAcβ1-3]₂β-SpNH-LC-LC; and Neu5Acα2-3[GalNAcβ1-4]Galβ1-4GlcNAcβ-SpNH-LC-LC.

In a further embodiment of the kit, (i) the further binding member comprises a first conjugating moiety capable of specifically conjugating with a second conjugating moiety; and (ii) the kit further comprises (c) a biosensor device comprising the second conjugating moiety operably bound to a zone on the surface of the biosensor device. For example, the first and second conjugating moieties can be selected from the group consisting of biotin, avidin, and streptavidin. T biosensor device can be a screen-printed carbon electrode (SPCE) or a membrane strip biosensor. In a refinement, (i) the further binding member is a glycan comprising a biotin moiety as the first conjugating member; (ii) the biosensor comprises streptavidin as the second conjugating member bound to the zone on the surface, and (iii) the glycan is immobilized on the surface of the biosensor by conjugation of the biotin moiety with the streptavidin moiety, for example further comprising gold nanoparticles (AuNP) at the surface to which the glycan is immobilized.

In another aspect, the disclosure relates to a biosensor device comprising a glycan as the second binding pair member in any if its various embodiments immobilized on a detection surface of the biosensor device.

In another aspect, the disclosure relates to a triplex comprising: (a) the biologically enhanced, electrically active magnetic (BEAM) nanoparticle composition according to any of its various embodiments, (b) a further binding pair member that specifically recognizes a subtype of the virus strain or the virus surface protein specifically recognized by the binding pair member of the BEAM nanoparticle composition, and (c) a virus or virally derived material comprising a virus strain or a virus surface protein, or a mutant or fragment thereof, wherein the virus or virally derived material is bound to both the binding pair member of the BEAM nanoparticle composition and the further binding pair member.

In another aspect, the disclosure relates to a method for detecting the presence of a virus strain or a virus surface protein in a sample, the method comprising: (a) providing the triplex according to any of its various embodiments, (b) detecting the triplex (e.g., by performing cyclic voltammetry to a biosensor device to which the triplex is immobilized), and, optionally (c) determining that the virus strain or the virus surface protein is present in the sample. For example, providing the triplex in part (a) can comprise: (i) immobilizing the further binding pair member on a surface; (ii) contacting the further binding pair with the sample for a time sufficient to bind any virus or virally derived material present in the sample to the further binding pair member, thereby forming a viral-further binding pair member conjugate; and (iii) contacting the viral-further binding pair member conjugate with the BEAM nanoparticle composition for a time sufficient to bind the binding pair member of the BEAM nanoparticle composition to the virus or virally derived material of the viral-further binding pair member conjugate, thereby forming the triplex immobilized on the surface. Alternatively, providing the triplex in part (a) can comprise: (i) contacting the further binding pair member and the BEAM nanoparticle composition with the sample for a time sufficient to bind any virus or virally derived material present in the sample to the further binding pair member and the binding pair member of the BEAM nanoparticle composition, thereby forming the triplex; and (ii) immobilizing the triplex on a surface. Alternatively, providing the triplex in part (a) can comprise: (i) immobilizing the further binding pair member on a surface; (ii) contacting the BEAM nanoparticle composition with the sample for a time sufficient to bind any virus or virally derived material present in the sample to the binding pair member of the BEAM nanoparticle composition, thereby forming a viral-BEAM nanoparticle conjugate; and (iii) contacting the a viral-BEAM nanoparticle conjugate with the further binding pair member for a time sufficient to bind the further binding pair member to the viral-BEAM nanoparticle conjugate, thereby forming the triplex immobilized on the surface. Alternatively, providing the triplex in part (a) can comprise: (i) contacting the further binding pair with the sample for a time sufficient to bind any virus or virally derived material present in the sample to the further binding pair member, thereby forming a viral-further binding pair conjugate; (ii) immobilizing the viral-further binding pair conjugate on a surface; and (iii) contacting the viral-further binding pair conjugate with the BEAM nanoparticle composition for a time sufficient to bind the binding pair member of the BEAM nanoparticle composition to the virus or virally derived material of the viral-further binding pair conjugate, thereby forming the triplex immobilized on the surface.

Various refinements of the disclosed method are possible. For example, the method can further comprise magnetically separating the triplex or a magnetic component thereof (e.g., a BEAM nanoparticle, a conjugate of the BEAM nanoparticle and the target virus/virally derived material, and/or the triplex itself) from a liquid medium (e.g., a liquid sample medium or otherwise) and concentrating the triplex or the magnetic component thereof (e.g., into a more concentrated liquid suspension and/or a dried material concentrate) prior to detecting the triplex in part (b). The sample can be saliva or serum obtained from a mammal (e.g., saliva or serum obtained from a human). The virus surface protein, or a mutant or fragment thereof, can be from a recombinant source. The virus or virally derived material comprising the virus strain or the virus surface protein, or a mutant or fragment thereof can be prepared from the sample, or contained in the sample. In an embodiment, detecting the triplex can comprise (i) acid-doping (e.g. doping with an acid such as HCl) the conductive polymer (e.g., polyaniline) of the triplex and then optionally (ii) performing cyclic voltammetry to a biosensor device to which the triplex is immobilized to detect the acid-doped triplex

All patents, patent applications, government publications, government regulations, and literature references cited in this specification are hereby incorporated herein by reference in their entirety. In case of conflict, the present description, including definitions, will control.

Additional features of the disclosure may become apparent to those skilled in the art from a review of the following detailed description, taken in conjunction with the examples, drawings, and appended claims, with the understanding that the disclosure is intended to be illustrative, and is not intended to limit the claims to the specific embodiments described and illustrated herein.

### BRIEF DESCRIPTION OF THE DRAWINGS

For a more complete understanding of the disclosure, reference should be made to the following detailed description and accompanying drawing wherein:

FIG. 1 illustrates a screen-printed carbon electrode (SPCE) testing schematic according to the disclosure: (a) SPCE consisting of two electrodes: carbon working electrode and silver/silver chloride counter/reference electrode, (b) stepwise preparation method, (c) preconcentration preparation method, and (d) schematic of the corresponding electrical circuit before and after analyte application.

FIG. 2 shows SPR results: (a) H5 140 nM binding to Biacore chip immobilized with H5-specific glycan 3′SLe^{x}, as inhibited by 1% mouse serum and α-H5 monoclonal antibody 1:500, (b) H5 140 nM binding to Biacore chip immobilized with H5-specific glycan 3′SLN, as inhibited by 1% mouse serum and α-H5 monoclonal antibody 1:500, (c) H5 140 nM binding to Biacore chip immobilized with H5-specific glycan 3′SLe^{x}, as inhibited by cross-reactivity of α-H1 polyclonal antibody; H5* 140 nM binding to 3′SLe^{x}, and (d) antibody testing on Biacore chip immobilized with H5-specific glycan 3′SLN: first injection, H5 at 140 nM for 10 min at 5 μl/min; second injection, α-H5 monoclonal antibody at 1:500 for 5 min at 5 μl/min.

FIG. 3 shows cyclic voltammetry results: (a) H5 concentration study as a function of preparation method and comparison to negative controls, as numbered and described in Table 2. Group (A) 1, (B) 2, (C) 3, (D) 24, (E) 25, (F) 27, (G) 26, (H) 9, (I) 10, (J) 11, (K) 14, (L) 13, (M) 12, (N) 15, (O) 16, (P) 17, (O) 18, and (R) 19. (b) Response for H5 1.4 μM using different preparation methods. (A) 1, (B) 8, (C) 9, (D) 20, and (E) 21. For the respective samples, mean ΔQ±SD, n=3 (SD=standard deviation, n=no. of replicates).

FIG. 4 shows TEM imaging data: (a) TEM and electron diffraction micrograph (inset) of EAM polyaniline nanoparticles with gamma iron (III) oxide cores, (b) TEM of EAMs immunofunctionalized with α-H5 antibody, (c) 3′SLe^{x}/H5/α-H5-EAM complex, magnetically separated and washed, with H5 prepared with 10% mouse serum, and (d) 3′SLe^{x}/H5/α-H5-EAM complex, magnetically separated and washed, with H5 prepared without serum.

FIG. 5 illustrates an investigation of SPCE sensitivity related to Glycan/H5/a-H5 mAb-EAM binding by the stepwise method.

FIG. 6 illustrates an investigation of SPCE sensitivity related to Glycan/H5/a-H5 mAb-EAM Binding by the preconcentration method.

FIG. 7 illustrates an investigation of SPCE sensitivity related to different preparation methods using a series of H5 samples.

FIG. 8 illustrates an investigation of SPCE sensitivity using a series of H1 samples.

FIG. 9 illustrates an investigation of SPCE sensitivity related to human pandemic detection using a series of H5 samples.

FIG. 10 illustrates an investigation of SPCE sensitivity related to human pandemic detection using a series of H1 samples.

While the disclosed compositions, kits, apparatus, and methods are susceptible of embodiments in various forms, specific embodiments of the disclosure are illustrated in the drawings (and will hereafter be described) with the understanding that the disclosure is intended to be illustrative, and is not intended to limit the claims to the specific embodiments described and illustrated herein.

### DETAILED DESCRIPTION

A particulate composition formed from a conductive polymer (e.g., conductive polyanilines) bound to magnetic nanoparticles (e.g., γ-Fe₂O₃) is disclosed. The particulate composition is alternatively referenced as an electrically-active magnetic (“EAM”) nanoparticle composition. The particulate composition can be formed into a biologically enhanced, electrically active magnetic (BEAM) nanoparticle composition by further including a binding pair member (e.g., an antibody or a fragment thereof that specifically recognizes a virus strain or a virus surface protein) bound to the conductive polymer of the particulate composition. In particular embodiments, the disclosure provides compositions, kits, detection apparatus, and methods for detecting specific viral strains including those with pandemic potential. In the various embodiments, a triplex including the BEAM, a virus or virally derived material (e.g. strain- and/or strain subtype specific viral surface protein or fragments thereof), and a viral strain subtype-specific binding pair member (e.g., a glycan that recognizes a specific virus strain subtype) is formed (e.g., with the three components conjugated or bound together), and the formation of said triplex is detected (e.g., by use of a biosensor). The disclosed compositions and methods are useful for the rapid, accurate, and selective detection of various viral pathogens (e.g., influenza hemagglutinin (HA) viral surface protein, such as any one of H1-H16), such as in assays exploiting the magnetic properties of the nanoparticle compositions (e.g., for analyte concentration) and using any of a variety of detection mechanisms (e.g., conductimetric detection, magnetic detection, using an enzyme label for colorimetric detection).

The BEAM nanoparticle composition can perform a dual function of a magnetic concentrator and a transducer in biosensing applications. The magnetic properties of the BEAM nanoparticles serve the purpose of concentrating and separating specific target analytes from complex sample matrices, while the electrical properties of the BEAM nanoparticles can be exploited in various detection schemes, for example biosensing applications which can be based on a conductimetric or other suitable type of assay.

These EAM nanoparticle compositions can mimic the function of magnetic beads widely used as a separator for immunomagnetic separation in immunoassays, for hybridization with nucleic acid probes as capture reagents, as templates in PCR, and the like. In addition, the electrical and the magnetic properties of the nanoparticles or composites can also be exploited as molecular transducers in biosensors. Some of the major advantages of the compositions include: (1) ability to perform the dual function of a magnetic concentrator as well as a biosensor transducer; (2) ability to achieve faster assay kinetics since the compositions are in suspension and in close proximity to target analytes; (3) increased surface area for the biological events to occur; (4) minimized matrix interference due to the improved separation and washing steps; (5) ability to magnetically manipulate the magnetic nanomaterials by using permanent magnets or electromagnets; (6) ability to avoid complex pre-enrichment, purification or pre-treatment steps necessary in standard methods of detection; (7) ability to design cheap, sensitive, highly specific and rapid detection devices for diverse targets by using different biological modifications; and (8) ability to design different rapid detection devices using both electrical and magnetic properties of the BEAM nanoparticles.

### Particulate Composition

The particulate compositions according to the disclosure generally include a conductive polymer bound to magnetic nanoparticles (e.g., a population of magnetic nanoparticles in which each nanoparticle generally has at least some conductive polymer bound thereto). U.S. Publication No. 2009/0123939, the entire contents of which are hereby incorporated herein by reference, discloses particulate compositions, biologically enhanced particulate compositions and related methods suitable for use according to the present disclosure.

The conductive properties of the conductive polymer (sometimes referenced as a synthetic metal) arise from the π-electron backbone and the single/double bonds of the π-conjugated system alternating down the polymer chain. Some conducting polymeric structures include polyaniline (PANi), polypyrrole, polyacetylene, and polyphenylene. Polyaniline, in particular, has been studied thoroughly because of its stability in fluid form, conductive properties, and strong bio-molecular interactions. Conductive polymers can be used in a biosensor, an analytical device capable of pathogen detection in which the conductive polymers act as electrochemical transducers to transform biological signals to electric signals that can be detected and quantified.

The conductive polymers according to the disclosure are not particularly limited and generally include any polymer that is electrically conductive. Preferably, the conductive polymer is fluid-mobile when bound to an analyte. Suitable examples of conductive polymers are polyanilines, polypyrrole, and polythiophenes, which are dispersible in water and are conductive because of the presence of an anion or cation in the polymer (e.g., resulting from acid-doping of the polymer or monomer). Other electrically conductive polymers include substituted and unsubstituted polyanilines, polyparaphenylenes, polyparaphenylene vinylenes, polythiophenes, polypyrroles, polyfurans, polyselenophenes, polyisothianapthenes, polyphenylene sulfides, polyacetylenes, polypyridyl vinylenes, biomaterials, biopolymers, conductive carbohydrates, conductive polysaccharides, combinations thereof and blends thereof with other polymers, copolymers of the monomers thereof. Conductive polyanilines are preferred. Polyaniline is perhaps the most studied conducting polymer in a family that includes polypyrrole, polyacetylene, and polythiophene. As both electrical conductor and organic compound, polyaniline possesses flexibility, robustness, highly controllable chemical and electrical properties, simple synthesis, low cost, efficient electronic charge transfer, and environmental stability. Addition of a protic solvent such as hydrochloric acid yields a conducting form of polyaniline, with an increase in conductivity of up to ten orders of magnitude. Illustrative are the conductive polymers described in U.S. Pat. Nos. 6,333,425, 6,333,145, 6,331,356 and 6,315,926. Preferably, the conductive polymers do not contain metals in their metallic form.

The conductive polymer provides a substrate for the subsequent attachment of a binding pair member bound thereto, which binding pair member is complementary to a target analyte and thereby forms a BEAM nanoparticle, as described below. The electrically conductive characteristics of the conductive polymer also can facilitate detection of an analyte bound to the BEAM nanoparticle, for example by measuring the electrical resistance or conductance through a plurality of BEAM nanoparticles immobilized in a capture region of conductimetric biosensor device. Additionally, an electrical current passing through plurality of BEAM nanoparticles can be used to induce a magnetic field, and properties such as magnetic permeability or mass magnetization can be detected and correlated to the presence of the target analyte in a sample.

The magnetic nanoparticles according to the disclosure are not particularly limited and generally include any nano-sized particles (e.g., about 1 nm to about 1000 nm) that can be magnetized with an external magnetic/electrical field. The magnetic nanoparticles more particularly include superparamagnetic particles, which particles can be easily magnetized with an external magnetic field (e.g., to facilitate separation or concentration of the particles from the bulk of a sample medium) and then redispersed immediately once the magnet is removed (e.g., in a new (concentrated) sample medium). Thus, the magnetic nanoparticles are generally separable from solution with a conventional magnet. Suitable magnetic nanoparticles are provided as magnetic fluids or ferrofluids, and mainly include nano-sized iron oxide particles (Fe₃O₄ (magnetite) or γ-Fe₂O₃ (maghemite)) suspended in a carrier liquid. Such magnetic nanoparticles can be prepared by superparamagnetic iron oxide by precipitation of ferric and ferrous salts in the presence of sodium hydroxide and subsequent washing with water. A suitable source of γ-Fe₂O₃ is Sigma-Aldrich (St. Louis, Mo.), which is available as a nano-powder having particles sized at <50 nm with a specific surface area ranging from about 50 m²/g to about 250 m²/g. Preferably, the magnetic nanoparticles have a small size distribution (e.g., ranging from about 5 nm to about 25 nm) and uniform surface properties (e.g., about 50 m²/g to about 245 m²/g).

More generally, the magnetic nanoparticles can include ferromagnetic nanoparticles (i.e., iron-containing particles providing electrical conduction or resistance). Suitable ferromagnetic nanoparticles include iron-containing magnetic metal oxides, for example those including iron either as Fe(II), Fe(III), or a mixture of Fe(II)/Fe(III). Non-limiting examples of such oxides include FeO, γ-Fe₂O₃ (maghemite), and Fe₃O₄ (magnetite). Other suitable magnetic core materials include, hydroxyl iron, and Li Ni ferrite, for example with hydrochloric acid, phosphoric acid, and toluene as doping agents. The magnetic nanoparticles can also be a mixed metal oxide of the type M1ₓM2₃₋ₓO₄, wherein M1 represents a divalent metal ion and M2 represents a trivalent metal ion. For example, the magnetic nanoparticles may be magnetic ferrites of the formula M1Fe₂O₄, wherein M1 represents a divalent ion selected from Mn, Co, Ni, Cu, Zn, or Ba, pure or in admixture with each other or in admixture with ferrous ions. Other metal oxides include aluminum oxide, chromium oxide, copper oxide, manganese oxide, lead oxide, tin oxide, titanium oxide, zinc oxide and zirconium oxide, and suitable metals include Fe, Cr, Ni or magnetic alloys.

The particulate composition is generally formed by the polymerization of a conductive polymer monomer (e.g., aniline, pyrrole) in a solution (e.g., aqueous) containing the magnetic nanoparticles. The polymerization solution generally includes an acid dopant (e.g., HCl) to impart electrical conductivity to the resulting polymer. The polymerization reaction is preferably initiated by the addition of an oxidant (e.g., ammonium persulfate). Upon completion of the polymerization reaction, the solution contains the particulate composition in which the resulting conductive polymer is bound to the magnetic nanoparticles. The magnetic nanoparticles and the monomer can be combined in any suitable weight ratio in the polymerization solution so that the resulting particulate composition has a desired balance of magnetic, electrical, and particle size properties. For example, the weight ratio of monomer:magnetic nanoparticles in the polymerization solution (or conductive polymer: magnetic nanoparticles in the resulting particulate composition) preferably ranges from about 0.01 to about 10, more preferably from about 0.1 to about 1 or about 0.4 to about 0.8, for example about 0.6. Similarly, the particulate composition preferably ranges in size from about 1 nm to about 500 nm, more preferably about 10 nm to about 200 nm or about 50 nm to about 100 nm.

### Biologically Enhanced, Electrically Active Magnetic Nanoparticles

Preferably, the particulate composition in any of its above embodiments is extended to a biologically enhanced, electrically active magnetic (BEAM) nanoparticle composition by further including a binding pair member bound to the conductive polymer of the particulate composition (e.g., directly or indirectly bound, with or without an intervening layer or linker between the conductive polymer and the binding pair member). The binding pair member is selected to be complementary to a target analyte so that the BEAM nanoparticle composition can be used for the selective detection of the target analyte in a sample.

An analyte (or target analyte) generally includes a chemical or biological material, including living cells, in a sample which is to be detected using the BEAM nanoparticle composition. The analyte can include pathogens of interest such as viral pathogens (e.g., influenza, such as influenza A (including serotypes/strains thereof), influenza B, influenza C) and/or bacterial pathogens (e.g., E. coli O157:H7, B. anthracis, and B. cereus). The analyte also may be an antigen, an antibody, a ligand (i.e., an organic compound for which a receptor naturally exists or can be prepared, for example one that is mono- or polyepitopic, antigenic, or haptenic), a single compound or plurality of compounds that share at least one common epitopic site, and a receptor (i.e., a compound capable of binding to an epitopic or determinant site of a ligand, for example thyroxine binding globulin, antibodies, enzymes, Fab fragments, lectins, nucleic acids, protein A, complement component C1q). In some embodiments, the term “analyte” also can include an analog of the analyte (i.e., a modified form of the analyte which can compete with the analyte for a receptor) that can also be detected using the BEAM nanoparticle composition.

In an embodiment, the analyte includes a viral pathogen, for example a virus or virally derived material such as a particular viral strain, a particular viral strain subtype, or a specific viral surface protein. Thus, the target analyte can be a selected virus from a class (e.g., genus or species) having more than one member, where the target analyte of interest can be differentiated from other members of the class. A virus of interest is the influenza virus, and the analyte can include any of influenza A (FLUAV; including and particular serotype/strain/subtype thereof), influenza B, or influenza C viruses or virally derived materials. Specific strains of FLUAV can be selected as the target analyte based on various surface protein combinations of hemagglutinin (HA) and neuraminidase (NA) for the strain. Any particular HA/NA surface protein alone or in combination can be selected, for example any combination of the sixteen known H1-H16 HA surface proteins (e.g., H1, H2, H3, H5, H7, H9, or H10) and/or the nine known N1-N9 NA surface proteins (e.g., N1, N2, N3, N4, N7, N8, N9). Thus, the analyte can be any desired FLUAV strain denoted HₓN_{y}, where x can be selected to have any desired value between 1 and 16 and y can be selected to have any desired value between 1 and 9 (e.g., H1N1, H1N2, H2N2, H3N1, H3N2, H3N8, H5N1, H5N2, H5N3, H5N8, H5N9, H7N1, H7N2, H7N3, H7N4, H7N7, H9N2, or H10N7 as relevant common strains).

A sample generally includes an aliquot of any matter containing, or suspected of containing, the target analyte. For example, samples can include biological samples, such as samples from taken from animals (e.g., saliva, whole blood, serum, plasma, urine, tears, and the like, such as from mammals or humans more particularly), cell cultures, plants; environmental samples (e.g., water); and industrial samples. Samples may be required to be prepared prior to analysis according to the disclosed methods. For example, samples may require extraction, dilution, filtration, centrifugation, and/or stabilization prior to analysis. For the purposes herein, “sample” can refer to either a raw sample as originally collected or a sample resulting from one or more preparation techniques applied to the raw sample.

The binding pair member (or specific binding partner) generally includes one of two different molecules, each having a region or area on its surface or in a cavity that specifically binds to (i.e., is complementary with) a particular spatial and polar organization of the other molecule. The binding pair members can be referenced as a ligand/receptor (or antiligand) pair. These binding pair members include members of an immunological pair such as antigen-antibody. Other specific binding pairs such as biotin-avidin (or derivatives thereof such as streptavidin or neutravidin), hormones-hormone receptors, IgG-protein A, polynucleotide pairs (e.g., DNA-DNA, DNA-RNA), DNA aptamers, and whole cells are not immunological pairs, but can be used as binding pair members within the context of the present disclosure.

Preferably, the binding pair members are specific to each other and are selected such that one binding pair member is the target analyte of interest and the other binding pair member is the constituent bound to the conductive polymer of the particulate composition. Binding specificity (or specific binding) refers to the substantial recognition of a first molecule for a second molecule (i.e., the first and second members of the binding pair), for example a polypeptide and a polyclonal or monoclonal antibody, an antibody fragment (e.g., a Fv, single chain Fv, Fab′, or F(ab′)₂ fragment) specific for the polypeptide, enzyme-substrate interactions, and polynucleotide hybridization interactions. Preferably, the binding pair members exhibit a substantial degree of binding specificity and do not exhibit a substantial amount of non-specific binding (i.e., non-covalent binding between molecules that is relatively independent of the specific structures of the molecules, for example resulting from factors including electrostatic and hydrophobic interactions between molecules).

Substantial binding specificity refers to an amount of specific binding or recognition between molecules in an assay mixture under particular assay conditions. Substantial binding specificity relates to the extent that the first and second members of the binding pair to bind only with each other and do not bind to other interfering molecules that may be present in the analytical sample. The specificity of the first and second binding pair members for each other as compared to potential interfering molecules should be sufficient to allow a meaningful assay to be conducted for the target analyte. The substantial binding specificity can be a function of a particular set of assay conditions, which includes the relative concentrations of the molecules, the time and temperature of an incubation, etc. For example, the reactivity of one binding pair member with an interfering molecule as compared to that with the second binding pair member is preferably less than about 25%, more preferably less than about 10% or about 5%.

A preferred binding pair member is an antibody (an immunoglobulin) that specifically binds to and is thereby defined as complementary with a particular spatial and polar organization of another molecule (e.g., an antigen). Antibodies generally include Y-shaped proteins on the surface of B cells that specifically bind to antigens such as bacteria, viruses, etc. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b, IgG3, IgM, etc. Fragments thereof may include Fab, Fv and F(ab′)₂, and Fab′. In addition, aggregates, polymers, and conjugates of immunoglobulins or their fragments can be used where appropriate so long as binding affinity for a particular molecule is maintained.

In an embodiment, the binding pair member bound to the conductive polymer is an antibody or antibody fragment that specifically recognizes a virus strain or virus surface protein. Thus, the binding pair member can specifically bind to a viral analyte with the selected strain or surface protein, and the binding pair member can differentiate from among at least some other members of the target analyte's genus or species. For example, the binding pair member can specifically bind to a hemagglutinin surface protein (e.g., influenza hemagglutinin HA) belonging to a particularly selected serotype, such as any one of the FLUAV HA serotypes H1-H16 (e.g., any one of H1, H2, H3, H4, H5, H6, H7, H8, H9, H10, H11, H12, H13, H14, H15, H16). In another embodiment, For example, the binding pair member can specifically bind to a neuraminidase surface protein (e.g., influenza neuraminidase NA) belonging to a particularly selected serotype, such as any one of the FLUAV NA serotypes N1-N9 (e.g., any one of N1, N2, N3, N4, N5, N6, N7, N8, and N9). Antibodies and antibody fragments specific to a particular surface protein can be monoclonal or polyclonal and can be obtained commercially or prepared by techniques that are well known in the art. Examples of two commercially obtainable antibodies include polyclonal anti-influenza virus H1 hemagglutinin (HA) protein (available from Immune Technology Corp. (New York, N.Y.)) and monoclonal anti-influenza virus H5 hemagglutinin (HA) protein (VN04-2) (available from NIH Biodefense and Emerging Infections Research Resources Repository, NIAID, NIH).

The binding pair member that is specific to the target analyte can be bound to the conductive polymer of the particulate composition by any of a variety of methods known in the art appropriate for the particular binding pair member (e.g., antibody, DNA oligonucleotide). For example, antibodies can be bound to the conductive polymer of the particulate composition by incubating the antibodies in a buffer (e.g., a phosphate buffer at a pH of about 7.4 containing dimethylformamide and lithium chloride) suspension of the particulate composition. Similarly, oligonucleotides can be incubated in a buffer (e.g., an acetate buffer at a pH of about 5.2) suspension of the particulate composition that also includes an immunoconjugating agent (e.g., 1-ethyl-3-(3-dimethylaminopropyl) carbodiimide hydrochloride (“EDAC”)). After a suitable incubation period (i.e., depending on the rate of binding between the binding pair member and the conductive polymer) the resulting BEAM nanoparticles can be blocked, washed, centrifuged, and then stored as a suspension (e.g., in aqueous LiCl for an antibody on a phosphate-buffered saline (“PBS”) solution for an oligonucleotide).

### Additional Binding Pair Member

The methods, compositions, and apparatus according to the disclosure generally include an additional binding pair member that is separate from the binding pair member bound to the conductive polymer of the BEAM nanoparticles (e.g., the binding pair member of the BEAM nanoparticle is the “first” binding pair member and the additional binding pair member is the “second” binding pair member). The second binding pair member can be different from the first binding pair member, but the two complement each other in the sense that the second binding pair member specifically recognizes a subtype of the virus strain or the virus surface protein specifically recognized by the first binding pair member of the BEAM nanoparticle composition. In use, the first binding pair member and the second binding pair member are capable of simultaneously or sequentially binding a virus strain or a virus surface protein, thereby forming a triplex comprising the first binding pair member of the BEAM nanoparticle and the second binding pair member bound to the virus strain or the virus surface protein. In an embodiment, the subtype has receptor specificity (e.g., conferring at least one of human infectivity and human to human transmissibility to the virus strain) for a host cell glycan receptor with terminal sialic acids dependent upon the linkage of the sialic acid to a saccharide moiety on the receptor.

While the particular nature of the second binding pair member is not particularly limited (e.g., such as the general binding pair members described above in relation to the BEAM nanoparticles), the second binding pair member is suitably a glycan (e.g., an oligosaccharide with at least 2 or 3 and/or up to 6, 8, or 10 monosaccharide residues) that preferentially binds host cell glycan receptors (e.g., the target analyte of BEAM nanoparticle, or analyte to which first binding pair member is specific). The glycan generally includes one or more terminal sialic acid saccharide residues (e.g., 1, 2, or 3 terminal sialic acid residues such as N-acetylneuraminic acid (Neu5Ac), or more generally other N- or O-substituted derivatives of neuraminic acid). The sialic acid residues are suitably linked by α2,6-, or α2,8-linkages, either with other sialic acid residues (e.g., which can be interior, non-terminal residues) or with non-sialic acid saccharide residues. In an embodiment, the glycan includes a terminal Neu5Ac residue with an α2,6-linkage to other interior monosaccharide residues. Examples of other monosaccharide residues for the glycan include N-acetylglucosamine (GlcNAc), galactose (gal), glucose (glu), and fucose (fuc), with corresponding examples of specific glycan second binding pair members including: Neu5Acα2-3Galβ1-4[Fucα1-3]GlcNAcβ-SpNH-LC-LC-Biotin; Neu5Acα2-3Galβ1-4GlcNAcβ-SpNH-LC-LC-Biotin; Neu5Acα2-8Neu5Acα2-8Neu5Acα2-3Galβ1-4Glcβ-SpNH-LC-LC-Biotin; Neu5Acα2-6Galβ1-4GlcNAcβ-SpNH-LC-LC-Biotin; Neu5Acα2-3Galβ1-4[Fucα1-3]GlcNAcβ-SpNH-LC-LC; Neu5Acα2-3Galβ1-4GlcNAcβ-SpNH-LC-LC; Neu5Acα2-8Neu5Acα2-8Neu5Acα2-3Galβ1-4Glcβ-SpNH-LC; Neu5Acα2-6Galβ1-4GlcNAcβ-SpNH-LC-LC; Neu5Acα2-3Galβ1-4[Fucα1-3]GlcNAcβ-Osp-LC-LC; Neu5Acα2-3Galβ1-4[Fucα1-3]GlcNAcβ-Osp-LC-LC-Biotin; Neu5Acα2-3[Galβ1-4GlcNAcβ1-3]₂β-SpNH-LC-LC-Biotin; Neu5Acα2-6[Galβ1-4GlcNAcβ1-3]₂β-SpNH-LC-LC-Biotin; Neu5Acα2-3[GalNAcβ1-4]Galβ1-4GlcNAcβ-SpNH-LC-LC-Biotin; Neu5Acα2-3Galβ1-4[Fucα1-3]GlcNAcβ-Osp-LC-LC; Neu5Acα2-3[Galβ1-4GlcNAcβ1-3]₂β-SpNH-LC-LC; Neu5Acα2-6[Galβ1-4GlcNAcβ1-3]₂β-SpNH-LC-LC; and Neu5Acα2-3[GalNAcβ1-4]Galβ1-4GlcNAcβ-SpNH-LC-LC.

As illustrated in the foregoing list, the glycan can include components other than monosaccharide residues. For example, the glycan can include spacer and/or conjugating moieties to facilitate the further functionalization or attachment of the glycan/second binding pair member to a substrate (e.g., such as a sensor surface) or other molecule. Such conjugating moieties are suitably located at a terminal end of the glycan (e.g., at the opposing end of the glycan chain relative to the terminal sialic acid). The specific conjugating moieties are not particularly limited as long as they have the ability to specifically bind with a complementary conjugating moiety, suitable examples of which include avidin, biotin, streptavidin, and neutravidin.

In an embodiment, the glycan/second binding pair member includes the conjugating moiety (e.g., a “first” conjugating moiety) capable of specifically conjugating with a second conjugating moiety, for example for use with a biosensor device that includes the second conjugating moiety operably bound to a zone on the surface of the biosensor device (e.g., a detection surface of the biosensor, where the surface can further include gold nanoparticles (AuNP) at the surface to which the second conjugating moiety is bound and the second binding pair member is immobilized). For example, the biosensor can be a screen-printed carbon electrode (SPCE) biosensor (e.g., where the second conjugating moiety is bound to a surface on the working electrode adjacent/between the counter/reference electrodes), a membrane strip biosensor (e.g., as in a lateral flow assay where the second conjugating moiety is bound to a capture region of the biosensor, for example between two electrodes), or a surface plasmon resonance (SPR) device (e.g., where the second conjugating moiety is bound to the SPR detection surface exhibiting resonance). Illustrative SPCE and SPR biosensor embodiments are illustrated in the examples below. Examples of suitable membrane strip/lateral flow biosensors are disclosed in U.S. Publication Nos. 2003/0153094 and 2008/0314766, both of which are incorporated herein in their entireties. Thus, in an embodiment, the disclosure provides a biosensor device with a glycan immobilized on the detection surface of the biosensor device (e.g., a glycan second binding pair member immobilized on the detection surface via a link between the first and second conjugating moieties). As a particular example, the second binding member can be a glycan with a biotin moiety as the first conjugating member; the biosensor can include streptavidin as the second conjugating member bound to the zone on the biosensor detection surface, and the glycan is immobilized on the surface of the biosensor by conjugation of the biotin moiety with the streptavidin moiety.

### Applications of BEAM Nanoparticles

The BEAM nanoparticles (which include the first binding pair member) and the second binding pair member from any of the above embodiments can be used in an assay to detect the presence of a target analyte such as a virus strain or a virus surface protein in a sample (e.g., saliva or serum obtained from a mammal such as human saliva or serum). The BEAM nanoparticles and/or and the second binding pair member can be provided in a variety of forms, for example a liquid suspension, a powder, or as part of an assay device (e.g., in an application region or capture region of a lateral flow assay device, on an electrode surface of an SPCE biosensor).

The method generally involves the formation of a triplex composition between: (a) a BEAM nanoparticle, (b) the second binding pair member, and (c) a virus or virally derived material (e.g., including a virus strain or a virus surface protein, or a mutant or fragment thereof), where the virus or virally derived material is bound to both the first binding pair member of the BEAM nanoparticle and the further binding pair member by specific binding pair interactions. The contact time required to obtain sufficient binding between the triplex components generally depends on the kinetics of the particular analyte-binding pair member interaction. However, sufficient contact times are generally short, for example less than about 60 minutes, more preferably ranging from about 2 or 5 minutes to about 10, 20, or 30 minutes. Suitable incubation temperatures can range from about 0° C. to 50° C., about 20° C. to 30° C., or about 25° C. Contact/incubation times and temperatures can be regulated directly in a reaction vessel. Suitably, the triplex is maintained under conditions that maintain the binding of the first binding member and the second binding member bound to the target analyte virus strain or said virus surface protein). Preferably, the triplex is immobilized on a biosensor surface for detection (e.g., by application of cyclic voltammetry), although the triplex can be formed first and then immobilized on the biosensor surface, or the triplex can be directly formed in an immobilized state on the biosensor surface.

In some embodiments, a magnetic field can be applied to a sample to concentrate the triplex present in the sample. Specifically, the applied magnetic field attracts the magnetic nanoparticle portion of the triplex (or a conjugate of the BEAM nanoparticle and analyte, but without the second binding pair member), causing individual particles of the triplex/conjugate to migrate to and concentrate in a region of the liquid medium containing the triplex/conjugate. Thus, after migration of the triplex/conjugate, a portion of the sample that is substantially free from the triplex/conjugate can be removed (e.g., by washing, draining, skimming, pipetting, etc.), thereby forming a sample concentrate that contains substantially all of the analyte triplex/conjugate (e.g., potentially in addition to any free BEAM nanoparticles). Preferably, at least about 80 wt. % to about 90 wt. % of the triplex/conjugate is recovered in the sample concentrate. Similarly, the concentration factor (i.e., the ratio of the concentration of the analyte-nanoparticle complex in the sample concentrate as compared to the original sample) is at least about 5, more preferably in the range of about 10 to about 50. If desired, the sample concentrate can then be processed for subsequent analyte detection.

The sample (or sample concentrate) is then analyzed to detect the presence of the triplex. A positive identification of the triplex in the sample (concentrate) indicates the presence of the target viral analyte in the original sample. If a quantitative determination of the triplex is made, any dilution and concentration factors can be used to determine the concentration of the target analyte in the original sample. The specific method of detection of the triplex is not particularly limiting, and can include methods applicable to immunoassays in general or immunomagnetic assays in particular (e.g., conductimetric detection, magnetic detection, agglomeration, spectrophotometric detection, colorimetric detection, radioactive detection, visual inspection, such as with an additional conventional label for non-conductimetric and non-magnetic detections).

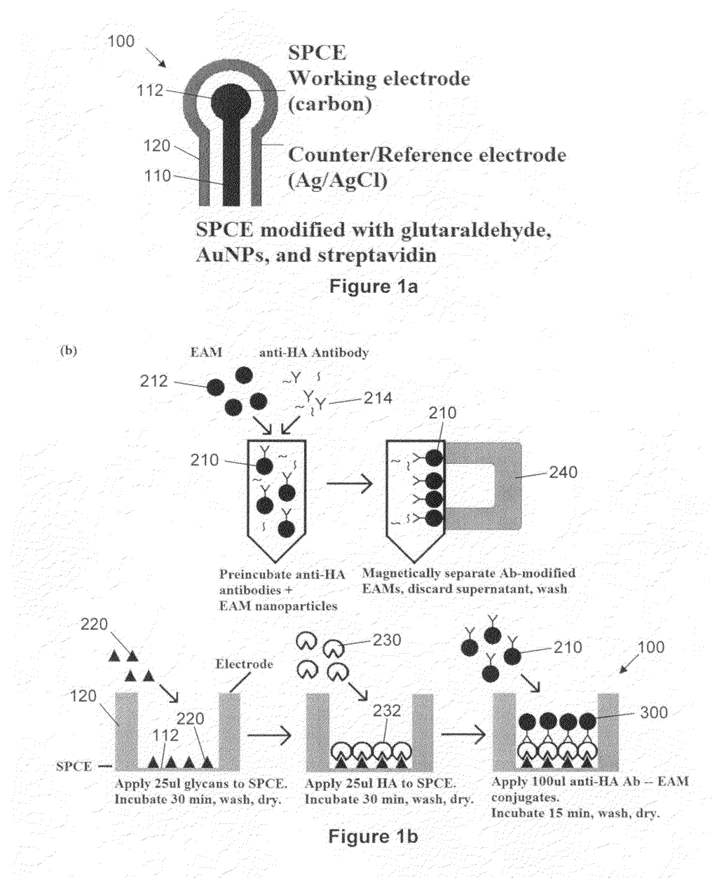

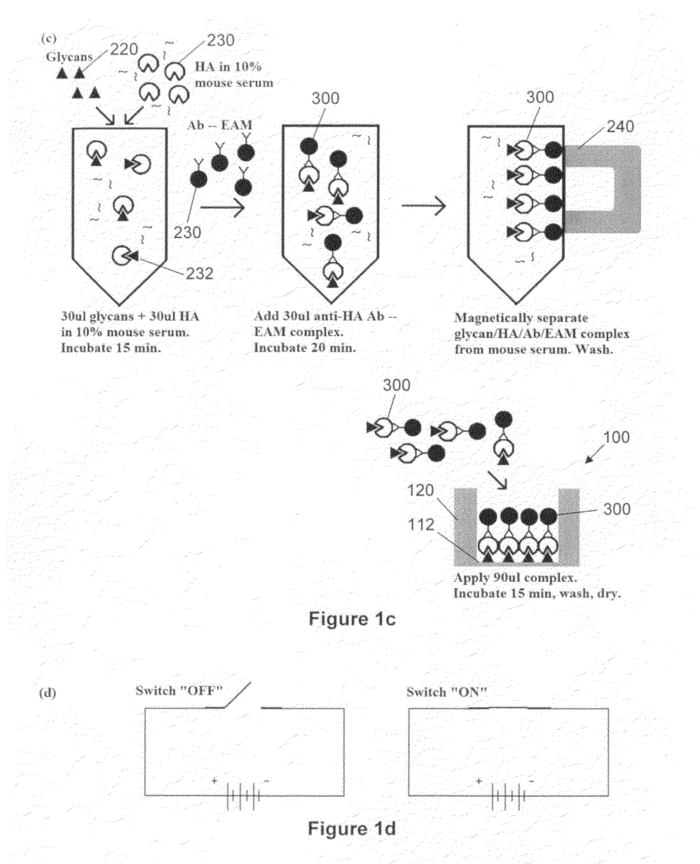

The specific order or triplex formation is not particularly limited, and various method embodiments are illustrated in FIGS. 1a-1d. FIG. 1a illustrates a biosensor 100 (an SPCE as shown) for detection of a target analyte viral triplex 300. The biosensor 100 includes a working electrode 110 and a counter/reference electrode 120. The working electrode 110 includes a functionalized surface 112 (e.g., including glutaraldehyde, AuNPs, streptavidin as described in the examples) for immobilization of the triplex. As illustrated in the examples, glutaraldehyde, AuNPs, and streptavidin can be used for immobilization, although any convenient method known in the art for immobilization of the second binding pair member such as a glycan can be used (e.g., non-specific adsorption, specific/conjugate binding, covalent substrate attachment).

The top half of FIG. 1b illustrates a general method for forming BEAM nanoparticles 210 according to the disclosure. An antibody 214 (or a fragment thereof) that specifically recognizes a virus strain or a virus surface protein is incubated in a suspension with EAM nanoparticles 212 (i.e., the particulate composition with a conductive polymer bound to magnetic nanoparticles) for a time sufficient to bind the antibody 214 to the conductive polymer of the EAM nanoparticles 212. The resulting BEAM nanoparticles 210 can then be magnetically separated and concentrated from the suspension by using a magnet 240, discarding any suspension supernatant, and then washing the BEAM nanoparticles 210.

As shown in the bottom half of FIG. 1b, the BEAM nanoparticles 210 can be used in a stepwise method to form and detect the target analyte viral triplex 300. In a first step, the second binding pair member 220 (e.g., a glycan) is immobilized on the functionalized surface 112 of the biosensor 110, for example by incubating a suspension containing the second binding pair member 220 above the functionalized surface 112 for a time sufficient to result in adsorption or conjugate binding between the two (e.g., binding between an immobilized streptavidin on the functionalized surface 112 and a terminal biotin on the glycan binding pair member 220). A sample containing a target analyte virus 230 (or virally derived material) is then contacted with the immobilized second binding pair member 220 for a time sufficient to bind any target virus 230 present in the sample to the second binding pair member 220 to form a viral-second binding pair member conjugate 232 immobilized on the biosensor 100 surface 112, for example by incubating a liquid sample containing the target virus 230 above the immobilized second binding pair member 220, followed by washing and drying the biosensor surface. The BEAM nanoparticle composition 210 is then contacted with the immobilized viral-second binding pair member conjugate 232 the for a time sufficient to bind the first binding pair member 214 of the BEAM nanoparticle 210 to the virus 230 of the viral-second binding pair member conjugate 232 to form the target analyte viral triplex 300 immobilized on the biosensor 100 surface 112, for example by incubating a liquid suspension containing the BEAM nanoparticles 210 above the viral-second binding pair member conjugate 232, followed by washing (e.g., to remove any non-conjugated BEAM nanoparticles 210) and drying the biosensor surface. Cyclic voltammetry can then be performed on the biosensor 100 to determine whether the triplex 300 is present (i.e., and whether thus the target analyte 230 was present in the original sample).

FIG. 1c illustrates a related preconcentration method to form and detect the target analyte viral triplex 300. The second binding pair member 220 and the BEAM nanoparticle composition 210 are contacted with the sample for a time sufficient to bind any target virus 230 present in the sample to the second binding pair member 220 and the first binding pair member 214 of the BEAM nanoparticle composition 210 to form the target analyte viral triplex 300 suspended in a liquid medium, for example by incubating the components in a liquid medium that can be the same or different from the liquid sample medium (e.g., the components can be mixed together in the liquid sample medium itself, or the sample and other components can be added to a separate liquid medium). The target analyte viral triplex 300 can then be magnetically separated and concentrated from the suspension by using a magnet 240, discarding any suspension supernatant, and then washing the triplex 300. The unbound triplex 300 is then immobilized on the functionalized surface 112 of the biosensor 110, for example by incubating a suspension containing the triplex 300 above the functionalized surface 112 for a time sufficient to result in adsorption or conjugate binding between the two (e.g., between an immobilized streptavidin on the functionalized surface 112 and a terminal biotin on the glycan binding pair member 220 of the triplex), followed by washing (e.g., to remove any non-conjugated BEAM nanoparticles 210 remaining from the previous preconcentration step) and drying the biosensor surface.

As further shown in FIG. 1c, the second binding pair member 220, the BEAM nanoparticle composition 210, and the target virus 230/sample need not be combined/contacted in a single step. As shown, in a first step, the second binding pair member 220 and the target virus 230/sample are contacted/incubated to form the viral-second binding pair member conjugate 232 described above in relation to FIG. 1b. In a second step, the BEAM nanoparticle composition 210 (e.g., as described above in relation to FIG. 1b) is contacted/incubated with the viral-second binding pair member conjugate 232 to form the triplex 300.

The foregoing techniques in FIGS. 1b and 1c are illustrative, and the components of the triplex 300 can be bound together and/or immobilized on a (biosensor) surface in any convenient manner. For example, in a method similar to that of FIG. 1b, the triplex can be formed by (i) immobilizing the second binding pair member on a surface, (ii) contacting the BEAM nanoparticle composition with the sample for a time sufficient to bind any virus or virally derived material present in the sample to the first binding pair member of the BEAM nanoparticle composition, thereby forming a viral-BEAM nanoparticle conjugate, and (iii) contacting the a viral-BEAM nanoparticle conjugate with the second binding pair member for a time sufficient to bind the second binding pair member to the viral-BEAM nanoparticle conjugate, thereby forming the triplex immobilized on the surface. Similarly, in another embodiment, the triplex can be formed by (i) contacting the second binding pair with the sample for a time sufficient to bind any virus or virally derived material present in the sample to the second binding pair member, thereby forming a viral-second binding pair conjugate, (ii) immobilizing the viral-second binding pair conjugate on a surface, and (iii) contacting the viral-second binding pair conjugate with the BEAM nanoparticle composition for a time sufficient to bind the first binding pair member of the BEAM nanoparticle composition to the virus or virally derived material of the viral-second binding pair conjugate, thereby forming the triplex immobilized on the surface.

As described above, the triplex can be detected once immobilized on an electrode surface of an SPCE biosensor. Any suitable biosensor platform may be used, however. For example, a sample containing a target virus-BEAM nanoparticle conjugate can be applied to a capture region of a lateral flow assay device, where the capture region includes an immobilized second binding pair member (e.g., a glycan adsorbed onto a membrane or conjugated/bound thereto such as with biotin/streptavidin). The sample can be applied to the capture region in a variety of ways, such as by direct addition thereto or by capillary transport of the sample from an application region to the capture region. The immobilized second binding pair member in the capture region retains the target virus-BEAM nanoparticle conjugate complex in the capture region and forms the immobilized triplex. The presence of the target analyte in the sample can be determined (e.g., and optionally quantified) by magnetically or conductimetrically detecting the triplex (i.e., the magnetic nanoparticle or conductive polymer component thereof) in the capture region, inasmuch as BEAM nanoparticles that are not bound to target analyte are transported by capillary action out of the capture region (e.g., into an absorption region of the device).

The disclosed compositions also can be provided in a variety of kits. In one embodiment, a kit includes a container (e.g., glass vial, ampule) that contains any of the various compositions according to the disclosure, for example as a powder or in a liquid suspension. More specifically, the composition can include EAM nanoparticles 212 in a non-biologically enhanced form (i.e., a conductive polymer bound to magnetic nanoparticles) so that a user can functionalize the composition with any desired binding first pair member to customize the composition to any desired target analyte. In another embodiment, the composition is a BEAM nanoparticle composition 210 with a first binding pair member that is an antibody or a fragment thereof that specifically recognizes a target virus strain or virus surface protein. The kit further includes the second binding pair member 220 as an additional composition relative to the nanoparticles 210/212. The kit also can include a reaction vessel (i.e., a container for mixing the compositions and a sample to be analyzed), and/or a biosensor 100 according to any of the disclosed embodiments. The kit 200 can generally include a variety of other optional components that may be desired and/or appropriate, for example a magnet, wash reagents, positive and/or negative control reagents, assay kit instructions, and other additives (e.g., stabilizers, buffers). The relative amounts of the various reagents may be varied widely, to provide for concentrations in solution of the reagents that substantially optimize the sensitivity of the assay. Particularly, the reagents may be provided as dry powders (e.g., lyophilized) which on dissolution will provide for a reagent solution having the appropriate concentrations for combining with the sample.

### EXAMPLES

The following examples illustrate various compositions, apparatus, and methods according to the disclosure for detecting specific viral pathogens, but are not intended to limit the scope of the claims appended hereto.

The examples illustrate the use of electrically active magnetic (EAM) polyaniline nanostructures as both a magnetic concentrator and signal transducer in a biosensor for the rapid detection of emerging pandemic FLUAV strains. The design utilizes EAM nanoparticles as the electrical transducer and monoclonal antibodies as the biological sensing element on a screen-printed carbon electrode (SPCE) platform. SPCEs consist of a carbon working electrode and silver reference electrodes printed on low-cost polymer backing (FIG. 1a), and offer high sensitivity, portability, and affordability. These single-use SPCEs are applicable on-site and offer reproducibility and reliability. The examples illustrate a method of magnetic separation, direct electrical detection, and immunochemistry in the development of a direct-charge transfer biosensor for the detection of FLUAV HA with speed, sensitivity, and specificity. In the examples, binding between purified recombinant HA and purified synthetic carbohydrate receptor is investigated and mimics the biosensor platform.

Electrically active magnetic (EAM) nanoparticles, consisting of aniline monomer polymerized around gamma iron (III) oxide (γ-Fe₂O₃) cores, serve as the basis of a direct-charge transfer biosensor developed for detection of surface glycoprotein hemagglutinin (HA) from the Influenza A virus (FLUAV) H5N1 (A/Vietnam/1203/04). H5N1 preferentially binds to α2,3-linked host glycan receptors. The EAM nanoparticles were immunofunctionalized with antibodies against target HA. In a stepwise addition method, synthetic glycans mimicking the host influenza receptor were incubated on screen-printed carbon electrodes (SPCEs), followed by incubation with recombinant HA and then anti-HA-EAM complexes. In a preconcentration preparation method, glycans were preincubated with HA prepared in 10% mouse serum and subsequently incubated with anti-HA-EAM complexes. The anti-HA-EAM complexes were shown to effectively act as immunomagnetic separator and concentrator of HA from mouse serum matrix. In both methods, the EAM nanoparticles served as the biosensor transducer. The polyaniline was made electrically active by hydrochloric acid doping and cyclic voltammetry was performed at a scan rate of 55 mV/sec from −0.4 to 1V, with four consecutive 2 min scans recorded. Preconcentration method offered a more robust response. Experimental results indicate that the biosensor is able to detect recombinant H5 HA at a concentration of 1.4 uM in 10% mouse serum. The biosensor showed high specificity for H5 as compared to H1 (H1N1 A/South Carolina/1/18). This novel design applies EAM nanoparticles as the immunomagnetic concentrator and signal transducer in a sensitive, specific, affordable, and easy-to-use biosensor with applications in disease monitoring and biosecurity.

Glycans, HA, and Antibodies: The biotinylated carbohydrate compounds 3′SLex (B157), 3′SLN (B84), GT3 (B108), and 6′SLN (B87) (summarized in Table 1 below along with other commercially available glycans) were provided by the Carbohydrate Synthesis/Protein Expression Core of The Consortium for Functional Glycomics funded by the National Institute of General Medical Sciences grant GM62116. The following reagent was obtained through the NIH Biodefense and Emerging Infections Research Resources Repository, NIAID, NIH: H5 Hemagglutinin (HA) Protein from Influenza Virus, A/Vietnam/1203/04 (H5N1), Recombinant from baculovirus, NR-10510 (Source A H5, referred to as H5). The following reagent was obtained through the NIH Biodefense and Emerging Infections Research Resources Repository, NIAID, NIH: Monoclonal Anti-Influenza Virus H5 Hemagglutinin (HA) Protein (VN04-2), A/Vietnam/1203/04 (H5N1), (ascites, Mouse), NR-2728. 6×His tagged H5 hemagglutinin (HA) protein from 293 cell culture, A/Vietnam/1203/04 (H5N1) (Source B H5, referred to as H5*); C-terminal 6×His tagged H1 hemagglutinin (HA) protein from 293 cell culture, A/South Carolina/1/18 (H1N1); and polyclonal anti-influenza virus H1 hemagglutinin (HA) protein, H1N1/Pan, (rabbit), were purchased from Immune Technology Corp. (New York, N.Y.).

TABLE 1
Saccharide sequences and predicted binding to H5N1

| | | Predicted to |
|---|---|---|
| Saccharide Name, Synthetic Spacer | Common Name | Bind H5N1 |
| Neu5Acα2-3Galβ1-4[Fucα1-3]GlcNAcβ-SpNH-LC-LC-Biotin | 3′Slex | Yes |
| Neu5Acα2-3Galβ1-4GlcNAcβ-SpNH-LC-LC-Biotin | 3′SLN | Yes |
| Neu5Acα2-3[Galβ1-4GlcNAcβ1-3]₂β-SpNH-LC-LC-Biotin | 3′S-Di-LN | Yes |
| Neu5Acα2-8Neu5Acα2-8Neu5Acα2-3Galβ1-4Glcβ-SpNH-LC-LC-Biotin | GT3 | No |
| Neu5Acα2-6Galβ1-4GlcNAcβ-SpNH-LC-LC-Biotin | 6′SLN | No |
| Neu5Acα2-6[Galβ1-4GlcNAcβ1-3]₂β-SpNH-LC-LC-Biotin | 6′S-Di-LN | No |
| Neu5Acα2-3[GalNAcβ1-4]Galβ1-4GlcNAcβ-SpNH-LC-LC-Biotin | CT/Sda | No |

Chemicals and Reagents: All solutions and buffers used in the biosensor study were prepared in de-ionized (DI) water (from Millipore Direct-Q system). Iron (III) oxide (γ-Fe₂O₃) nanopowder, aniline monomer, ammonium persulfate, hydrochloric acid (HCl), methanol, diethyl ether, hydrogen tetrochloroaurate (III) trihydrate, sodium citrate dehydrate, glutaraldehyde, Polysorbate-20 (Tween-20), phosphate buffered saline (PBS), trizma base, casein, sodium phosphate (dibasic and monobasic), and streptavidin were purchased from Sigma-Aldrich (St. Louis, Mo.). Solutions were prepared as follows: PBS buffer (10 mM PBS, pH 7.4), wash buffer (10 mM PBS, pH 7.4, with 0.05% Tween-20), phosphate buffer (100 mM phosphate buffer, pH 7.4), casein blocking buffer (100 mM Tris-HCl buffer, pH 7.6, with 0.1% w/v casein), and glycine blocking buffer (67 μM glycine in 10 mM PBS, pH 7.4). HBS-P buffer, 10 mM glycine pH 2.5, and 50 mM NaOH were purchased from GE Healthcare (Piscataway, N.J.). Avidin/Biotin blocking kit was purchased from Vector Laboratories, Inc. (Burlingame, Calif.). Mouse serum (ICR SCID) was purchased from Bioreclamation, Inc. (Liverpool, N.Y.).

### Example 1

### Synthesis of BEAM Nanoparticles

EAM Polyaniline Nanostructure Synthesis: Aniline monomer was polymerized around gamma iron (III) oxide (γ-Fe₂O₃) cores to obtain magnetic/polyaniline core/shell (c/s) nanoparticles (Sharma et al., 2005). Commercially manufactured γ-Fe₂O₃ nanoparticles were sonicated and dispersed in 50 ml of 1M HCl, 10 ml deionized (DI) water, and 0.4 ml aniline monomer at 0° C. for 1 h. The γ-Fe₂O₃:monomer weight ratio was fixed at 1:0.6. 1 g ammonium persulfate in 20 ml DI water was added as oxidant while the mixture was stirred at 0° C. As electrically-active polyaniline, typically green, was formed over the γ-Fe₂O₃ nanoparticles, typically brown, the color of the solution visibly transitioned from rust brown to dark green. The reaction proceeded for 4 h with continuous stirring at 0° C. The solution was filtered, washed with 1M HCl, 10% methanol, and diethyl ether, and dried for 18 h. The resulting green solid was ground into fine powder and stored in a vacuum desiccator. The electrically-active magnetic/polyaniline core/shell nanoparticles have been previously characterized in terms of structure, size, magnetization, and conductivity (Pal et al., 2008a; Pal and Alocilja, 2009).

EAM Immunofunctionalization/BEAM Nanoparticle Formation: EAM nanoparticles were immunofunctionalized with either α-H5 monoclonal antibody IgG2 or α-H1 polyclonal antibody to form the resulting BEAM nanoparticles, as generally illustrated in FIG. 1b. Desiccated EAM polyaniline nanoparticles were dissolved in 100 mM phosphate buffer (pH 7.4) to obtain a concentration of 10 mg/ml, and sonicated for 15 min. The EAM polyaniline nanoparticles were then conjugated with α-H5 monoclonal antibodies by direct physical adsorption as previously described and confirmed by Pal and Alocilja (2009). α-H5 monoclonal antibody IgG2 (mouse ascites fluid) or α-H1 polyclonal antibody (rabbit) was added to the EAM polyaniline nanoparticles to obtain an antibody:EAM ratio of 1:10 by volume. The solution was incubated for 1 h at 25° C. in a rotational hybridization oven (Amerex Instruments, Inc., Lafayette, Calif.). Following adsorption of antibody onto the EAM nanoparticles, the immunofunctionalized nanoparticles were magnetically separated using a FlexiMag Magnetic Separator (Spherotech, Inc., Lake Forest, Ill.) to remove any unbound antibody in the supernatant. The anti-HA-EAM complexes were washed twice with blocking buffer consisting of 100 mM tris-HCl buffer (pH 7.6) with 0.1% (w/v) casein with magnetically separated supernatant discarded each time. The anti-HA-EAM complexes were then resuspended in 100 mM phosphate buffer (pH 7.4). The anti-HA-EAM complexes were prepared on the day of testing and stored at 4° C. until use.

### Example 2

### SPCE Biosensor Fabrication and Testing

SPCE Modification: SPCE chips were prepared by removing the overlaying mesh and foam (Gwent, Inc., UK). Each chip was washed with 2 ml sterile DI water and air dried for 15 min. As described in Lin et al. (2008), 25 μl of 2.5 mM glutaraldehyde solution were applied to the working area and incubated at 4° C. for 1 h. The SPCEs were then washed with 2 ml DI water and air dried at 25° C. for 15 min. 25 μl of AuNP solution were applied to the glutaraldehyde-treated working electrode and incubated at 4° C. for 1 h. The SPCEs were then washed with 2 ml DI water and air dried at 25° C. for 15 min. 20 μl of streptavidin at 1 μg/ml were applied to the working area and dried at 4° C. for 2 h or overnight.

Gold Nanoparticle Synthesis: Application of the carbon-based glycans directly onto the screen-printed carbon electrode would result in an insulating device. To enhance the electron transducer, thus amplifying response current and improving detection limits, gold nanoparticles (AuNPs) were applied to the SPCEs (Daniel and Astruc, 2004; Lin et al., 2008; Willner et al., 2007). AuNPs were synthesized according to a published procedure and their size, spectroscopic properties, and magnetic profiles have been previously characterized (Hill and Mirkin, 2006; Zhang et al., 2009). The referenced synthesis procedure required hydrogen tetrochloroaurate (III) trihydrate aqueous solution (1 mM, 50 mL) to be stirred while heated. Vigorous reflux was achieved, followed by titration with 5 mL of 38.8 mM sodium citrate. The solution shifted from yellow to the deep red characteristic of the AuNPs.

Preconcentration Technique—Sample Preparation and Capture: Glycans were prepared at 3× desired concentration in 0.01M PBS. HAs were prepared at 3× desired concentration in 0.01M PBS with 10% mouse serum (ICR SCID) by volume. 30 μl each of glycan and HA were incubated for 15 min at 25° C. in a rotational hybridization oven. 30 μl of the appropriate anti-HA-EAM complex was then added to the glycan/HA solution and incubated for 20 min at 25° C. in a rotational hybridization oven. The glycan/HA/anti-HA-EAM complexes were magnetically separated and washed twice with 0.01M PBS containing 0.05% Tween-20 for 5 minutes and resuspended in 0.01M PBS. The SPCE chips prepared with glutaraldehyde, AuNPs, and streptavidin were then treated with the biotinylated glycan/HA/anti-HA-EAM complex. 90 μl of the solution was applied to the treated SPCE and incubated at 25° C. for 15 min. The SPCE was washed with 2 ml DI water and air dried at 25° C. for 15 min (FIG. 1c).

Stepwise Technique—Sample Preparation and Capture: 25 μl of the desired glycan concentration were added to the working area of the glutaraldehyde, AuNPs, and streptavidin treated electrode and allowed to incubate at 25° C. for 30 min. Excess was rinsed with 2 ml DI water and air dried at 25° C. for 15 min. Available sites were blocked with sequential additions of 25 μl Avidin D and biotin solutions for 30 min each, with DI water rinse and air dry after each. 25 μl of the desired H5 concentration were added, incubated at 25° C. for 30 min, rinsed with 2 ml DI water, and air dried at 25° C. for 15 min. 100 μl of anti-HA-EAM complex solution were added to the electrode, incubated at 25° C. for 15 min, rinsed with 2 ml DI water, and air dried at 25° C. for 15 min (FIG. 1b).

Biosensor Detection and Data Analysis: Cyclic voltammetric measurements were performed using a 263A potentiostat/galvanostat (Princeton Applied Research, MA, USA) connected to a personal computer. Data collection and analysis were controlled through the PowerSuite electrochemical software operating system (Princeton Applied Research, Wellesley, Mass.). SPCE chips purchased from Gwent Inc. (UK) are shown in FIG. 1a.

100 μl of 0.1M HCl solution were applied to cover the entire SPCE electrode area and allowed to incubate for 5 min to acid-dope the polyaniline of the EAM particles. The SPCE electrodes were connected to the potentiostat and cyclic voltammetry was performed at a scan rate of 55 mV/sec and a cyclic scan range of −0.4 to 1V, with four consecutive 2 min scans recorded. Previous experimentation indicated that the third scan produced the most pronounced current flow differences for different samples and was chosen for analysis. For each experiment, including positive and negative controls, three replications were performed. The samples were calibrated against a negative control, also repeated in triplicate, which consisted of the anti-HA-EAM application step alone. The total charge transferred, ΔQ, was computed from the cyclic voltammogram as the integral of current, according to the relationship

I=ΔQ/Δt (1)

where I=current (A), ΔQ=charge transferred (C), and Δt=time elapsed (s) (Kuznetsov, 1995). The ΔQ values described in this paper were calculated from the current and time interval data generated by the potentiostat. Standard deviations and mean ΔQ values of the third scans for the triplicate data sets were calculated.

The presence of the target is indicated by an increase in total charge transferred across the electrodes. Target HA labeled with the immunofunctionalized EAMs were captured on the SPCE surface, and the EAMs, consisting of conductive polyaniline synthesized around a magnetic γ-Fe2O3 core, formed an electrical circuit between the silver electrodes, with current recorded by the potentiostat (FIG. 1d).

The lowest detection limit of the biosensor for H5 was investigated. The prepared biosensors were tested using three samples at 1:2 dilution in 0.01M PBS to obtain H5 at 100 μg/ml, 50 μg/ml, and 25 μg/ml. Testing for each dilution was performed in triplicate. Anti-HA-EAM complexes without glycan or HA were tested as the control. The lowest dilution of H5 that produced a signal distinguishable from the control was taken as the sensitivity of detection.

The specificity of the biosensor was investigated using H1, α-H1, and glycans nonspecific for H5, GT3 (α2,8 binder) and 6′SLN (α2,6 binder). The H1 was prepared at 1.4 μM in 0.01M PBS, the non-H5 binding glycans were prepared at 100 μM, and the EAMs were immunofunctionalized with α-H1 at 1:10 as described in Example 1.

The complexity of biological samples was considered, as the ultimate application of the biosensor as an in-field detection system would require testing of blood or sputum samples. In the preconcentration method, the HA samples were prepared to consist of 10% mouse serum (e.g., to mimic potential matrix interference effects of an actual serum sample). After complexing the glycan/HA/anti-HA-EAM, the magnetic separation and washing technique was investigated for its ability to specifically isolate the target HA from a complex serum matrix.

Each sample preparation was tested in triplicate with the biosensors to nullify the effect of equipment or user variation. The prepared biosensors were assumed to have the same physical properties. The mean and standard deviations of the ΔQ values were calculated for each sample preparation, including negative controls. The differences between the means were calculated and analyzed based on single factor analysis of variance (ANOVA) to a significance of 95% (P<0.05). The effects of different HAs, glycans, α-HA antibodies, and HA concentration were assessed to calculate the lower detection limit of the biosensor as well as the biosensor specificity.

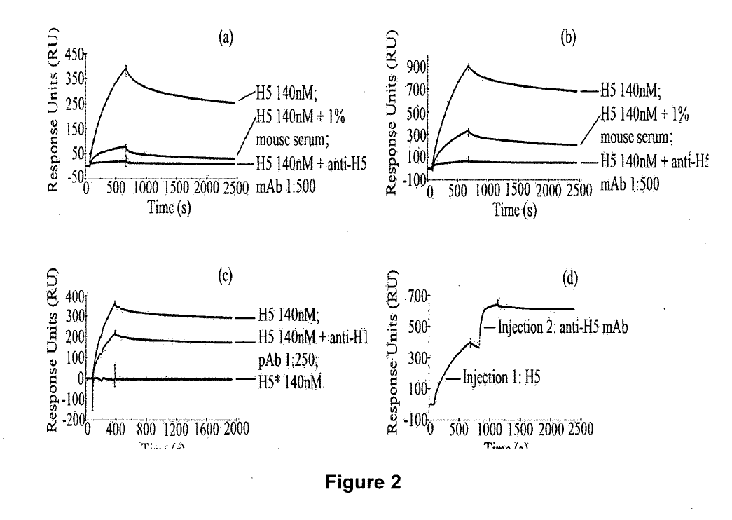

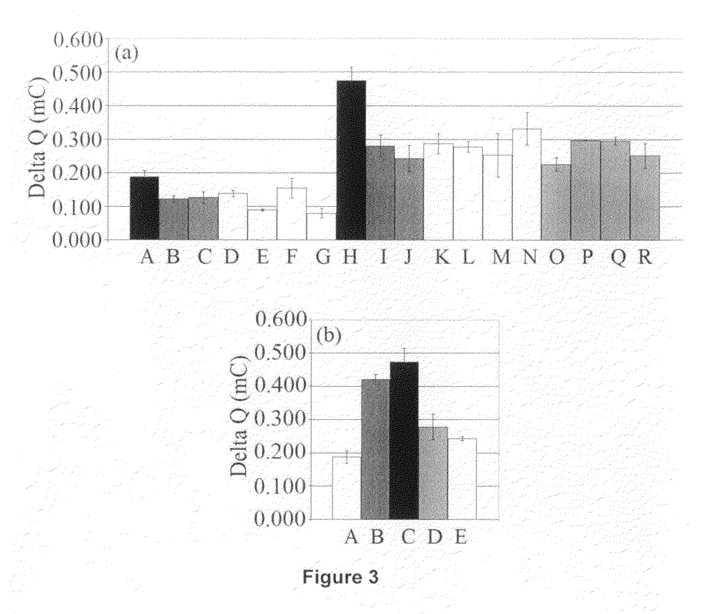

SPCE Results: The biosensor platform showed correlation to the SPR assay results. The sensitivity of the biosensor platform was explored by testing a range of H5 concentrations. The preconcentration preparation method yielded an average ΔQ value of 0.474 mC for the H5 at 1.4 μM binding to 3′SLe^{x}. The lower concentrations of 700 nM and 360 nM displayed significantly decreased ΔQ values which were not statistically different from each other or from the negative controls (FIG. 3a (H-J), Table 2). The stepwise preparation method yielded an average ΔQ value of 0.188 mC for the H5 at 1.4 μM, which was within the range of the preconcentration method negative controls and was statistically lower than the preconcentration value for H5 1.4 μM (Table 2). The lower H5 concentrations of the stepwise method were not statistically different from each other but also showed significantly lower ΔQ values than the 1.4 μM stepwise (FIG. 3a (A-C), Table 2). This would indicate that the preconcentration method, which includes two magnetic separation and wash steps, is better able to isolate the target HA, thus offering a consistently higher ΔQ value than the equivalent concentrations prepared using the stepwise method. The preconcentration HA preparations also included 10% mouse serum, which the stepwise HA did not, but the increased signal for the preconcentration method is not likely attributable to nonspecific binding due to the mouse serum. It can be observed that the preconcentration method when performed with the same concentrations of glycan and H5 with and without 10% mouse serum yielded similar ΔQ values, though still statistically different (P=0.0324) (FIG. 3b (B,C), Table 2). It is a likely conclusion then that the magnetic separation technique was able to fully extract the target HA from the 10% mouse serum matrix to yield a similar signal to that obtained when the sample was prepared with no serum. This is an improvement on the SPR assay, in which 1% mouse serum depressed the signal (FIG. 2a,b).

TABLE 2
Average signal of the H5 biosensor for samples tested by stepwise or preconcentration method

| | | | Mean ΔQ ± S.D. |
|---|---|---|---|
| | Preparation | | (n = 3) |
| # | Method | Reagents | (mC) |
| 1 | Stepwise | 3′SLe^{x} 100 μM − H5 1.4 μM − anti-H5-EAM | 0.18782 ± 0.0108^{a} |
| 2 | Stepwise | 3′SLe^{x} 100 μM − H5 700 nM − anti-H5-EAM | 0.12188 ± 0.0053^{b} |
| 3 | Stepwise | 3′SLe^{x} 100 μM − H5 360 nM − anti-H5-EAM | 0.12657 ± 0.0100^{b} |
| 4 | Stepwise | 3′SLe^{x} 100 μM − H1 1.4 μM − anti-H5-EAM | 0.08906 ± 0.0045^{b} |
| 5 | Stepwise | 3′SLe^{x} 100 μM − H1 1.4 μM − anti-H1-EAM | 0.11863 ± 0.0180^{b} |
| 6 | Stepwise | no glycan − H1 1.4 μM − anti-H1-EAM | 0.11747 ± 0.0227^{b} |
| 7 | Stepwise | no glycan − no HA − anti-H1-EAM | 0.13402 ± 0.0073^{b} |
| 8 | Preconc. | (3′SLe^{x} 100 μM + H5 1.4 μM) + anti-H5-EAM | 0.42050 ± 0.0087^{d} |
| 9 | Preconc. | [3′SLe^{x} 100 μM + (H5 1.4 μM + 10% mouse serum)] + anti-H5-EAM | 0.47444 ± 0.0230^{e} |
| 10 | Preconc. | [3′SLe^{x} 100 μM + (H5 700 nM + 10% mouse serum)] + anti-H5-EAM | 0.28153 ± 0.0188^{f} |
| 11 | Preconc. | [3′SLe^{x} 100 μM + (H5 360 nM + 10% mouse serum)] + anti-H5-EAM | 0.24322 ± 0.0226^{f} |
| 12 | Preconc. | [no glycan + (H5 1.4 μM + 10% mouse serum)] + anti-H5-EAM | 0.2533l ± 0.0373^{f} |
| 13 | Preconc. | (3′SLe^{x} 100 μM + no H5) + anti-H5-EAM | 0.27839 ± 0.0089^{f} |
| 14 | Preconc. | [GT3 100 μM + (H5 1.4 μM + 10% mouse serum)] + anti-H5-EAM | 0.28812 ± 0.0172^{f} |
| 15 | Preconc. | no glycan + no HA + anti-H5-EAM | 0.33222 ± 0.0281^{f} |
| 16 | Preconc. | [3′SLe^{x} 100 μM + (H1 1.4 μM + 10% mouse serum)] + anti-H5-EAM | 0.22594 ± 0.0115^{f} |
| 17 | Preconc. | [3′SLe^{x} 100 μM + (H1 1.4 μM + 10% mouse serum)] + anti-H1-EAM | 0.29739 ± 0.0001^{f} |
| 18 | Preconc. | [no glycan + (H1 1.4 μM + 10% mouse serum)] + anti-H1-EAM | 0.29573 ± 0.0068^{f} |
| 19 | Preconc. | no glycan + no HA + anti-H1-EAM | 0.25182 ± 0.0207^{f} |
| 20 | Preconc. | [3′SLe^{x} 100 μM + (H5* 1.4 μM + 10% mouse serum)] + anti-H5-EAM | 0.27883 ± 0.0222^{g} |
| 21 | Stepwise | 3′SLN 100 μM − H5 1.4 μM − anti-H5-EAM | 0.24382 ± 0.0037^{g} |
| 22 | Stepwise | 3′SLN 500 μM − H5 1.4 μM − anti-H5-EAM | 0.28515 ± 0.0479 |
| 23 | Stepwise | 3′SLN 500 μM − H5 290 nM − anti-H5-EAM | 0.24340 ± 0.0810 |
| 24 | Stepwise | 6′SLN 500 μM − H5 1.4 μM − anti-H5-EAM | 0.13880 ± 0.0056^{h} |
| 25 | Stepwise | 3′SLN 500 μM − noHA − anti-H5-EAM | 0.08950 ± 0.0014ⁱ |
| 26 | Stepwise | no glycan − noHA − anti-H5-EAM | 0.07957 ± 0.0080ⁱ |
| 27 | Stepwise | no glycan − H5 1.4 μM −− anti-H5-EAM | 0.15473 ± 0.0167^{h} |

S.D. = standard deviation and n = no. of replicates.
Mean ΔQ with different superscript letters (a through h) are significantly different at 95% confidence level (P < 0.05).

The signals generated for the same H5 concentration, 1.4 μM, were compared using different preparation methods. The preconcentration method, with or without 10% mouse serum added to the H5, yielded statistically higher ΔQ values than the stepwise method (FIG. 3b). The stepwise method did confirm that H5 binds to 3′SLN with statistically higher avidity than it binds to 3′SLe^{x}, which is confirmatory to SPR results (FIG. 2a,b). However, both of these stepwise values fell far lower than the preconcentration method values. Source A H5 was also shown to be a better binder to 3′SLe^{x} than Source B H5*. H5*, while the same strain as Source A H5, yielded a far lower ΔQ value when preconcentrated with 3′SLe^{x} than for the 3′SLe^{x}/H5 (Source A) preconcentration result (FIG. 3b (C,D)). However, 3′SLe^{x}/H5* preconcentration did yield a higher ΔQ value with statistical significance as compared to the 3′SLex/H5 (Source A) prepared stepwise (FIG. 3b (A,D), Table 2). Thus, the preconcentration method offers a more robust response and that H5 from Source A offers stronger binding to the 3′SLN and 3′SLex than H5*, possibly due to the predicted aggregated nature of Source A H5.

In both preparation methods, the negative controls yielded ΔQ values that were statistically lower than the reading from the H5-specific glycan/H5 interaction, with H5 at 1.4 μM and glycans 3′SLN or 3′SLe^{x}. For the stepwise preparation method, the presence of H5 at 1.4 μM, whether incubated after the nonbinder glycan 6′SLN or after no glycan, resulted in average ΔQ values lower with statistical significance than the 3′SLN/H5 response, but higher with statistical significance than the negative controls with no H5 added to either the H5-specific glycan or no glycan (FIG. 3a (A, D-G), Table 2). The absence of H5 yielded repeatable negative controls. The presence of H5 in those negative controls which resulted in higher ΔQ values than in those negative controls without H5 indicates that there may be low levels of nonspecific binding between H5 and the SPCE surface or any of the immobilized partners previously incubated on the SPCE. Further blocking could prove useful to eliminate nonspecific binding.

For the preconcentration method, the negative controls both with and without H5 were repeatable and within a statistically similar range (FIG. 3a (K-R), Table 2). The negative controls, including the GT3/H5 interaction, were also statistically lower than the 3′SLe^{x}/H5 interaction. The negative control which included no glycan and no HA but only the α-H5-EA, antibody complex yielded the highest ΔQ value of the negative controls, but this remained below the positive control (FIG. 3a (N), Table 2).

The preconcentration method did not include a blocking step, while in the stepwise method the SPCE surface was blocked with avidin and biotin after incubation with the biotinylated glycans or, when no glycan was included in the sample, before addition of HA or anti-HA-EAM complexes. The preconcentration method does not lend itself to blocking with avidin and biotin, since all of the interaction partners, including glycan, HA, and anti-HA-EAM are added simultaneously as an already formed complex. However, the lack of a blocking step does not appear to influence the signal with nonspecific binding effects. The magnetic separation step serves to eliminate irrelevant material which could interfere with target binding.

The specificity of the system was investigated using a series of H1 samples. In the preconcentration method, the H1, diluted to 1.4 μM with 10% mouse serum, was preincubated with the H5-specific glycan 3′SLe^{x} and subsequently incubated with EAMs conjugated with either α-H5 or α-H1 antibodies. The samples containing both H1 and α-H1-EAM complexes showed an increase in ΔQ as compared to the samples with no H1 or with α-H5-EAM complexes (FIG. 3a (O-R)). This may indicate that the H1 and α-H1 antibodies interact and cause slightly higher levels of nonspecific binding as compared to H1 alone or α-H1 alone. However, the levels of all H1-based negative controls remain within the statistical range of the H5-based negative controls (Table 2). This indicates that despite the polyclonal nature of the α-H1 antibodies, there is little cross-reactivity with the H5-targeted biosensor which improves upon the Biacore system (FIG. 2c). Both stepwise and preconcentration methods yielded ΔQ values for the binding to the H5-nonspecific glycans, GT3 or 6′SLN, which were distinguishably lower than their corresponding positive binder, 3′SLe^{x} or 3′SLN. Thus, the biosensor is highly specific for H5.

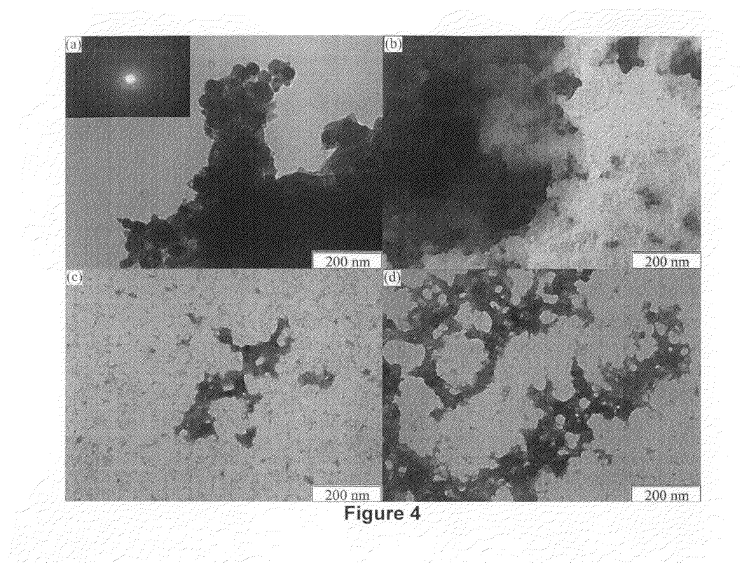

The EAM polyaniline nanoparticles, EAMs immunofunctionalized with α-H5 antibody, and glycan/HA/anti-HA-EAM complex were analyzed by a JEOL (Peabody, Mass.) 100CX II Transmission Electron Microscope (TEM) to obtain their structural morphologies. 1% uranyl acetate was used to stain α-H5 antibody, HA, and glycans. The crystalline nature of the EAM nanoparticles was also studied by selected area electron diffraction using the JEOL 2200FS field emission TEM. As shown in FIG. 4a, the TEM and electron diffraction micrograph revealed EAM polyaniline nanoparticle sizes in the 25-100 nm range. As observed in the TEM image, the darkest circular areas correspond to the γ-Fe₂O₃ cores which are surrounded by the lighter colored polyaniline polymerized around the cores. Immunofunctionalization of the EAM nanoparticles yields a cloudier border as compared to the crisp edge of the EAM nanoparticles alone, indicating that immunofunctionalization was effective (FIG. 4b). TEM imaging of the 3′SLe^{x}/H5/α-H5-EAM antibody complex after two magnetic separations and washes resulted in a web-like boundary which could be attributed to the binding of the H5 and glycan, forming a more branched complex than the EAMs or immunofunctionalized EAMs alone. When comparing the 3′SLe^{x}/H5/α-H5-EAM antibody complex prepared with H5 with and without 10% mouse serum, the TEM images reveal similarly shaped aggregates, indicating that there is no nonspecific binding of the serum components to the complex (FIG. 4c,d). This is in confirmation of the cyclic voltammetry results (FIG. 3b (B,C)). The backgrounds of the images do reveal that the sample prepared with mouse serum has a cloudier supernatant, suggesting the benefit of a more thorough washing, although the ΔQ values are not affected.

Preconcentration of target analyte is an option to extend the above method to optimize the biosensor in terms of sensitivity to detect HA at concentrations reflecting the viral load in an influenza infected patient. Nonspecific binding can be further reduced by improving blocking techniques or wash steps. The specificity, affordability, portability, and repeatability of the disclosed apparatus, compositions, and methods are promising. The biosensor design is easily adaptable to detection of other FLUAV strains, including the current swine-origin H1N1. The Biacore SPR (Example 3 below) assay is a complementary technique for understanding the specificity and avidity of glycan/HA partners and for probing cross-clade protection of α-HA antibodies.

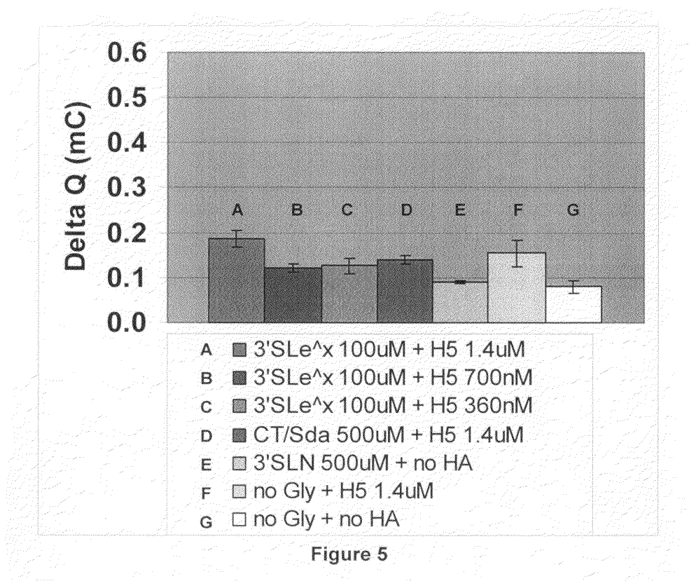

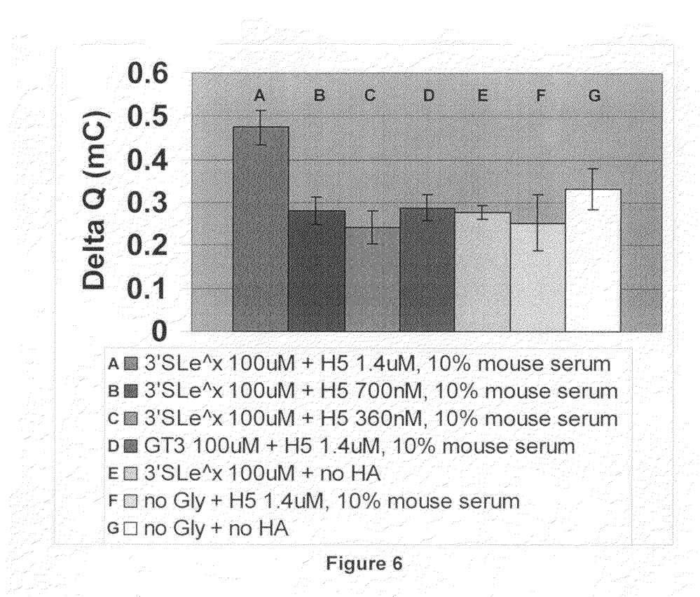

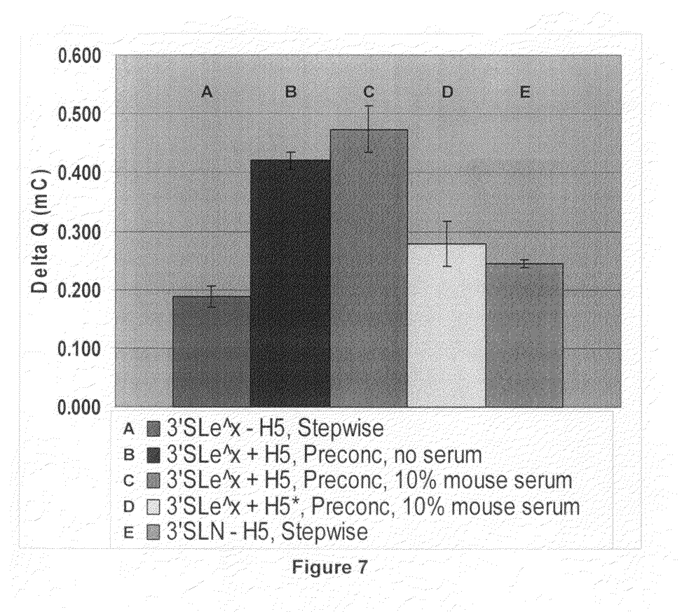

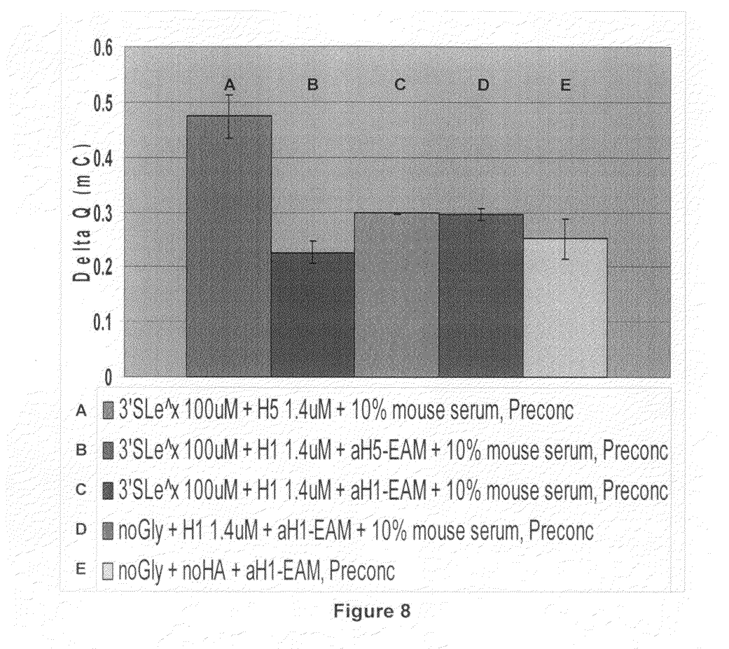

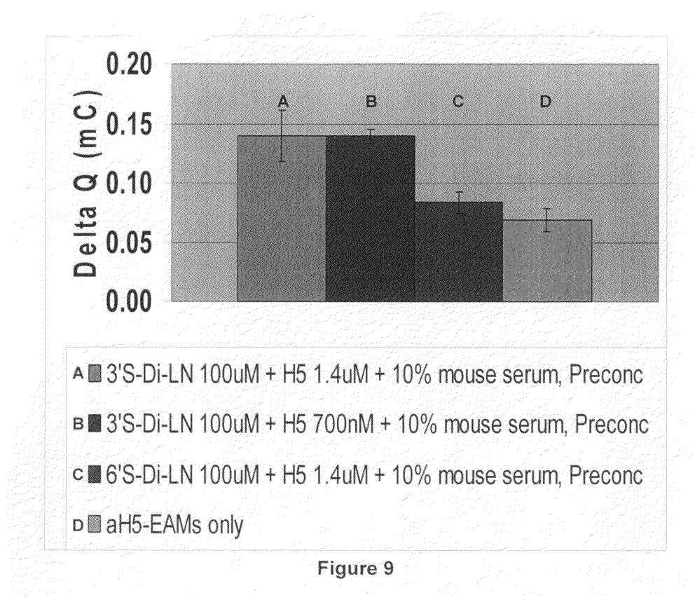

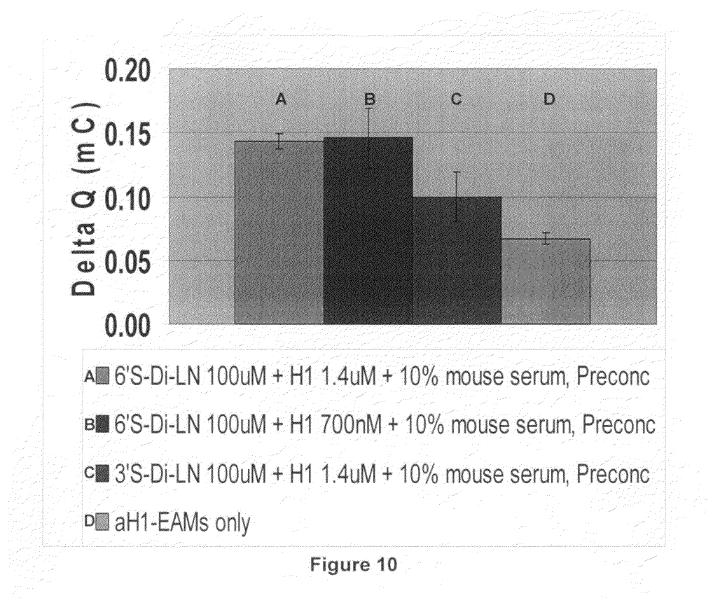

SPCE Comparative Results: Additional results illustrating the SPCE biosensor performance as shown in FIGS. 5-10. FIGS. 5 and 6 illustrate Glycan/H5/α-H5 mAb-EAM Binding by the stepwise (FIG. 5) and preconcentration (FIG. 6) methods. The preconcentration preparation method yielded an average ΔQ value of 0.474 mC for the H5 at 1.4 μM binding to 3′SLex. The lower concentrations of 700 nM and 360 nM displayed statistically lower ΔQ values. The stepwise preparation method yielded an average ΔQ value of 0.188 mC for the H5 at 1.4 μM, which was within the range of the preconcentration method negative controls and was statistically lower than the preconcentration value for H5 1.4 μM. The lower H5 concentrations prepared stepwise were not statistically different from each other but also showed significantly lower ΔQ values than the 1.4 μM stepwise. In both methods, negative controls were statistically lower than the H5-specific glycan/H5 interaction, with H5 at 1.4 μM and glycans 3′SLN or 3′SLex. In FIG. 7, signals generated for the same H5 concentration, 1.4 μM, were compared using different preparation methods. The stepwise method confirmed that H5 binds to 3′SLN with statistically higher avidity than it binds to 3′SLex, which is confirmatory to SPR results. The preconcentration method, with or without 10% mouse serum added to the H5, yielded statistically higher ΔQ values than the stepwise method. Higher signal for preconcentration is not likely due to nonspecific binding of the 10% mouse serum. The preconcentration method when performed with the same concentrations of glycan and H5 with and without 10% mouse serum yielded similar ΔQ values, though still statistically different (P=0.0324). FIG. 8 illustrates an investigation of SPCE sensitivity using a series of H1 samples. Negative controls with H1 and α-H1-EAM complexes showed an increase in ΔQ as compared to the samples with no H1 or with α-H5-EAM complexes. All H1-based negative controls remained within the statistical range of the H5-based negative controls. Both stepwise and preconcentration methods yielded ΔQ values for the binding to the H5-nonspecific glycans, GT3 or 6′SLN, which were distinguishably lower than their corresponding positive binder, 3′SLex or 3′SLN. In relation to human pandemic detection, FIGS. 9 and 10 illustrate that H5 binds α2,3-linked receptors with higher avidity than α2,6 glycans (FIG. 9), whereas H1 binds α2,6 glycans with higher avidity than α2,3 glycans (FIG. 10).

Conclusion: This example describes a design which utilizes electrically active magnetic nanoparticles both as a magnetic separator and a biosensor transducer. The biosensor system is rapid to results, with signal detection time at 10 minutes. The SPCEs may be prepared prior to testing and stored for 3 months. Using the preconcentration method, the entire sample preparation time requires 75 minutes, including complex incubation, magnetic separations, washes, and SPCE application. The sensitivity of the biosensor in the detection of recombinant H5 hemagglutinin (H5N1 A/Vietnam/1203/04) was found to be 1.4 μM in 10% mouse serum. The biosensor demonstrates high avidity of binding between H5 and α2,3-linked glycans 3′SLe^{x} and 3′SLN with statistically lower binding between H5 and α2,6-linked 6′SLN and α2,8-linked GT3, which is confirmatory to expected results and demonstrates that the biosensor can characterize HA by sialic acid receptor preference. The biosensor showed high specificity for H5 as compared to H1 (H1N1 A/South Carolina/1/18). The results indicate that the biosensor technology is valuable as a rapid, specific, and sensitive detection method with applicability at point-of-care for identifying pandemic avian influenza viruses with α2,3 specificity. From these results, the biosensor system should be easily modifiable to similarly detect HAs with α2,6 specificity, an indicator of human pandemic potential. The results demonstrate the ability of the EAMs to immunomagnetically separate target HA from serum matrix, and this capacity can be exploited in applications in which whole or pseudotyped virus is identified in complex matrices such as serum or respiratory secretions. The development of such a biosensor technology which identifies FLUAV HA based on specificity to host sialic acids is a significant initiative with applications in disease monitoring and homeland security.

### Example 3

### SPR Biosensor Fabrication and Testing

SPR Assay Design and Chip Preparation: Glycan partners were chosen for the HAs of interest based on widely accepted HA specificities, as previously investigated using glycan microarrays (Blixt et al., 2004; Stevens et al., 2006). Biotinylated glycans were diluted to 1 μg/ml in Biacore HBS-P buffer and 8 μl were injected over a Biacore Streptavidin (SA) chip at 10 μl/min. Glycans were immobilized to saturation at approximately 300 resonance units (Ru). H5 HA (Vietnam) at 140 nM was incubated with a serial dilution of α-H5 monoclonal antibody (shown to be neutralizing for H5 HA in standard hemagglutination inhibition assays) for 10 min at 25° C. and injected over the glycan chip surface to investigate the ability of the antibodies to neutralize the glycan/H5 binding. Binding was assessed by an increase in Ru. After 25 min dissociation time, the glycan surface was regenerated with 60 s of 10 mM glycine pH 2.5 and 18 s of 50 mM NaOH at 100 μl/min. The ability of the α-H5 monoclonal antibody to bind to the glycan/H5 complex was also investigated. H5 at 140 nM was injected over the immobilized glycans for 10 min at 5 μl/min. After 1 min dissociation and no regeneration, α-H5 monoclonal antibody was injected over the glycan/H5 complex for 5 min at 5 μl/min.

SPR Results: SPR analysis demonstrated a high avidity, specific binding between H5-specific α2,3-linked glycan receptors and recombinant H5. Preincubating H5 with neutralizing α-H5 monoclonal antibody results in a neutralization of glycan/H5 binding on the SPR system. α-H5 monoclonal antibody IgG2 (mouse ascites fluid) at 1:500 neutralized the binding between H5 at 140 nM and H5-specific glycans 3′SLe^{x} and 3′SLN (FIG. 2a,b, Table 1). The glycan/H5 binding showed slight inhibition by α-H1 polyclonal antibody at 1:250 but the α-H1 did not cause complete neutralization as observed at the same concentration by α-H5 (FIG. 2c).

The order of interaction was found to be important. The glycan/H5 binding was not neutralized when the same α-H5 monoclonal antibody was allowed to react with the already formed glycan/H5 complex. Following typical H5-binding, a further increased SPR signal indicated that the second injection of α-H5 monoclonal antibody also bound, forming a glycan/H5/α-H5 complex (FIG. 2d). The subsequently added α-H5 monoclonal antibody thus did not displace the glycan but instead bound the H5 in a region outside of the receptor binding domain or in an available binding domain if the H5 is present as a trimer or larger aggregate. This is in contrast to the neutralization experiment, in which the α-H5 monoclonal antibody binds within, or otherwise blocks, the glycan receptor binding domain on H5. This α-H5 monoclonal antibody is thus appropriate for use in both the SPR neutralization assay as well as the biosensor sandwich-type assay.

The SPR assay was also repeated with a 1% mouse serum matrix. Although binding was still observed, the results indicated that the glycan/H5 binding was inhibited by the addition of serum to the sample buffer (FIG. 2a,b).

Because other modifications and changes varied to fit particular operating requirements and environments will be apparent to those skilled in the art, the disclosure is not considered limited to the examples chosen for purposes of illustration, and covers all changes and modifications which do not constitute departures from the true spirit and scope of this disclosure.

Accordingly, the foregoing description is given for clarity of understanding only, and no unnecessary limitations should be understood therefrom, as modifications within the scope of the disclosure may be apparent to those having ordinary skill in the art.

Throughout the specification, where the compositions, kits, processes, or apparatus are described as including components, steps, or materials, it is contemplated that the compositions, processes, or apparatus can also comprise, consist essentially of, or consist of, any combination of the recited components or materials, unless described otherwise. Component concentrations expressed as a percent are weight-percent (% w/w), unless otherwise noted. Numerical values and ranges can represent the value/range as stated or an approximate value/range (e.g., modified by the term “about”). Combinations of components are contemplated to include homogeneous and/or heterogeneous mixtures, as would be understood by a person of ordinary skill in the art in view of the foregoing disclosure.

### REFERENCES

1. Amano, Y., Cheng, Q., 2005. Detection of influenza virus: traditional approaches and development of biosensors. Anal. Bioanal. Chem. 381, 156-164.
2. Blixt, O., Head, S., Mondala, T., Scanlan, C., Huflejt, M. E., Alvarez, R., Bryan, M. C., Fazio, F., Calarese, D., Stevens, J., Razi, N., Stevens, D. J., Skehel, J. J., van Die, I., Burton, D. R., Wilson, I. A., Cummings, R., Bovin, N., Wong, C.-H., Paulson, J. C., 2004. Printed covalent glycan array for ligand profiling of diverse glycan binding proteins. Proc. Natl. Acad. Sci. USA 101 (49), 17033-17038.
3. Daniel, M.-C., Astruc, D., 2004. Gold nanoparticles: assembly, supramolecular chemistry, quantum-size-related properties, and applications toward biology, catalysis, and nanotechnology. Chemical Reviews 104, 293-346.
4. Ellis, T. M., Bousfield, R. B., Bissett, L. A., Dyrting, K. C., Luk, G. S., Tsim, S. T., Sturm-Ramirez, K., Webster, R. G., Guan, Y., Peiris, J. S. M., 2004. Investigation of outbreaks of highly pathogenic H5N1 avian influenza in waterfowl and wild birds in Hong Kong in late 2002. Avian Pathol. 33, 492-505.
5. Faix, D. J., Sherman, S. S., Waterman, S. H., 2009. Rapid-test sensitivity for novel swine-origin Influenza A (H1N1) virus in humans. N. Engl. J. Med. 361 (7), 728-729.
6. Gurtler, L., 2006. Chapter 3: Virology of human Influenza, in: Kamps, B. S., Hoffmann, C., Preiser, W. (Eds.), Influenza Report 2006. Flying Publisher, Paris, pp. 48-86.
7. Hill, H. D., Mirkin, C. A., 2006. The bio-barcode assay for the detection of protein and nucleic acid targets using DTT-induced ligand exchange. Nature Protocols 1, 324-336.
8. Homeland Security Council (HSC), 2005. National strategy for pandemic Influenza. Available at: http://www.whitehouse.gov/homeland/nspi.pdf. Accessed 5 Apr. 2009.
9. Lin, Y.-H., Chen, S.-H., Chuang, Y.-C. Lu, Y.-C., Shen, T. Y., Chang, C. A., Lin, C.-S., 2008. Disposable amperometric immunosensing strips fabricated by Au nanoparticles-modified screen-printed carbon electrodes for the detection of foodborne pathogen Escherichia coli O157:H7. Biosens. Bioelectron. 23, 1832-1837.
10. Magalhaes, R. J. S., Ortiz-Pelaez, A., Thi, K. L. L., Dinh, Q. H., Otte, J., Pfeiffer, D. U., 2010. Associations between attributes of live poultry trade and HPAI H5N1 outbreaks: a descriptive and network analysis study in northern Vietnam. BMC Vet. Res. 6 (10), 1-10.
11. Meeusen, C., Alocilja, E. C., Osburn, W., 2005. Detection of E. coli O157:H7 using a miniaturized surface plasmon resonance biosensor. Transactions of the ASAE 48 (6), 2409-2416.
12. Michaelis, M., Doerr, H. W., Cinatl Jr., J., 2009. Novel swine-origin influenza A virus in humans: another pandemic knocking at the door. Med. Microbiol. Immunol. 198 (3), 175-183.
13. Muhammad-Tahir, Z., Alocilja, E. C., Grooms, D. L., 2007. Indium tin oxide-polyaniline biosensor: fabrication and characterization. Sensors Journal 7, 1123-1140.
14. Neumann, G., Chen, H., Gao, G. F., Kawaoka, Y., 2010. H5N1 influenza viruses: outbreaks and biological properties. Cell Research 20 (1), 51-61.
15. Pal, S., Alocilja, E. C., 2009. Electrically active polyaniline coated magnetic (EAPM) nanoparticle as novel transducer in biosensor for detection of Bacillus anthracis spores in food samples. Biosens. Bioelectron. 24, 1437-1444.
16. Pal, S., Alocilja, E. C., Downes, F. P., 2007. Nanowire labeled direct-charge transfer biosensor for detecting Bacillus species. Biosens. Bioelectron. 22, 2329-2336.
17. Pal, S., Setterington, E., Alocilja, E. C., 2008a. Electrically-active magnetic nanoparticles for concentrating and detecting Bacillus anthracis spores in a direct-charge transfer biosensor. IEEE Sensors Journal 8 (6), 647-654.
18. Pal, S., Ying, W., Alocilja, E. C., Downes, F. P., 2008b. Sensitivity and specificity performance of a direct-charge transfer biosensor for detecting Bacillus cereus in selected food matrices. Biosystems Engineering Journal 99 (4), 461-468.
19. Reid, A. H., Taubenberger, J. K., Fanning, T. G., 2001. The 1918 Spanish Influenza: integrating history and biology. Microbes Infect. 3, 81-87.
20. Stevens, J., Blixt, O., Glaser, L., Taubenberger, J. K., Palese, P., Paulson, J. C., Wilson, I. A., 2006. Glycan microarray analysis of the hemagglutinins from modern and pandemic Influenza viruses reveals different receptor specificities. J. Mol. Biol. 355, 1143-1155.
21. Sturm-Ramirez, K. M., Ellis, T., Bousfield, B., Bissett, L., Dyrting, K., Rehg, J. E., Poon, L., Guan, Y., Peiris, M., Webster, R. G., 2004. Reemerging H5N1 influenza viruses in Hong Kong in 2002 are highly pathogenic to ducks. J. Virol. 78 (9), 4892-4901.
22. Webster, R. G., Bean, W. J., Gorman, O. T., Chambers, T. M., Kawaoka, Y., 1992. Evolution and ecology of influenza A viruses. Microbiol. Rev. 56, 152-179.
23. Werner, 0., Harder, T. C., 2006. Chapter 2: Avian Influenza, in: Kamps, B. S.,
Hoffmann, C., Preiser, W. (Eds.), Influenza Report 2006. Flying Publisher, Paris, pp. 48-86.
24. Wiley, D. C., Skehel, J. J., 1987. The structure and function of the hemagglutinin membrane glycoprotein of Influenza virus. Ann. Rev. Biochem. 56, 365-94.
25. Willner, I., Baron, R., Willner, B., 2007. Integrated nanoparticle-biomolecule systems for biosensing and bioelectronics. Biosens. Bioelectron. 22, 1841-1852.
26. Wright, P. F., Webster, R. G., 2001. Orthomyxoviruses, in: Knipe, D. M., Howley, P. M. (Eds.), Fields Virology, 4th ed. Lippincott Williams & Wilkins, Philadelphia, pp. 1533-1579.
27. Zhang, Z., Wan, M., Wei, Y., 2005. Electromagnetic functionalized polyaniline nanostructures. Nanotechnology 16, 2827-2832.

What is claimed is:

1. A biologically enhanced, electrically active magnetic (BEAM) nanoparticle composition comprising:
(a) a particulate composition comprising a conductive polymer bound to magnetic nanoparticles; and
(b) a binding pair member bound to the conductive polymer of the particulate composition, wherein the binding pair member is an antibody or a fragment thereof that specifically recognizes a virus strain or a virus surface protein.

2. The BEAM nanoparticle composition of claim 1, wherein the binding pair member specifically recognizes a hemagglutinin serotype as the virus strain.

3. The BEAM nanoparticle composition of claim 1, wherein the binding pair member specifically recognizes and is capable of binding a hemagglutinin as the virus surface protein.

4. The BEAM nanoparticle composition of claim 3, wherein the binding pair member is an antibody that specifically recognizes an influenza B hemagglutinin virus surface protein selected from the group consisting of H1, H2, H3, and H5.

5. The BEAM nanoparticle composition of claim 1, wherein:
(i) the magnetic nanoparticles comprise at least one of Fe(II) and Fe(III); and,
(ii) the conductive polymer is selected from the group consisting of polyanilines, polypyrroles, polythiophenes, derivatives thereof, combinations thereof, blends thereof with other polymers, and copolymers of the monomers thereof.

6. A kit comprising:
(a) the biologically enhanced, electrically active magnetic (BEAM) nanoparticle composition of claim 1, and
(b) a further binding pair member that specifically recognizes a subtype of the virus strain or the virus surface protein specifically recognized by the binding pair member of the BEAM nanoparticle composition.

7. The kit of claim 6, wherein the further binding pair member is a glycan that preferentially binds host cell glycan receptors, the glycan comprising α2,6-, α2,3-, or α2,8-linked sialic acid.

8. The kit of claim 7, wherein the glycan further comprises a conjugating moiety selected from the group consisting of avidin, biotin, and streptavidin.

9. The kit of claim 6, wherein the further binding pair member is a glycan that preferentially binds host cell glycan receptors, the glycan comprising α2,6-linked sialic acid.

10. The kit of claim 6, wherein the further binding pair member is a glycan selected from the group consisting of:
Neu5Acα2-3Galβ1-4[Fucα1-3]GlcNAcβ-SpNH-LC-LC-Biotin;
Neu5Acα2-3Galβ1-4GlcNAcβ-SpNH-LC-LC-Biotin;
Neu5Acα2-8Neu5Acα2-8Neu5Acα2-3Galβ1-4Glcβ-SpNH-LC-LC-Biotin;
Neu5Acα2-6Galβ1-4GlcNAcβ-SpNH-LC-LC-Biotin;
Neu5Acα2-3Galβ1-4[Fucα1-3]GlcNAcβ-SpNH-LC-LC;
Neu5Acα2-3Galβ1-4GlcNAcβ-SpNH-LC-LC;
Neu5Acα2-8Neu5Acα2-8Neu5Acα2-3Galβ1-4Glcβ-SpNH-LC;
Neu5Acα2-6Galβ1-4GlcNAcβ-SpNH-LC-LC;
Neu5Acα2-3Galβ1-4[Fucα1-3]GlcNAcβ-Osp-LC-LC;
Neu5Acα2-3Galβ1-4[Fucα1-3]GlcNAcβ-Osp-LC-LC-Biotin;
Neu5Acα2-3[Galβ1-4GlcNAcβ1-3]₂β-SpNH-LC-LC-Biotin;
Neu5Acα2-6[Galβ1-4GlcNAcβ1-3]₂β-SpNH-LC-LC-Biotin;
Neu5Acα2-3[GalNAcβ1-4]Galβ1-4GlcNAcβ-SpNH-LC-LC-Biotin;
Neu5Acα2-3Galβ1-4[Fucα1-3]GlcNAcβ-Osp-LC-LC;
Neu5Acα2-3[Galβ1-4GlcNAcβ1-3]₂β-SpNH-LC-LC;
Neu5Acα2-6[Galβ1-4GlcNAcβ1-3]₂β-SpNH-LC-LC; and
Neu5Acα2-3[GalNAcβ1-4]Galβ1-4GlcNAcβ-SpNH-LC-LC.

11. The kit of claim 6, wherein the subtype has receptor specificity for a host cell glycan receptor with terminal sialic acids dependent upon the linkage of the sialic acid to a saccharide moiety on the receptor.

12. The kit of claim 11, wherein the receptor specificity confers at least one of human infectivity and human to human transmissibility to the virus strain.

13. The kit of claim 6, wherein:
(i) the further binding member comprises a first conjugating moiety capable of specifically conjugating with a second conjugating moiety; and
(ii) the kit further comprises (c) a biosensor device comprising the second conjugating moiety operably bound to a zone on the surface of the biosensor device.

14. The kit of claim 13, wherein the biosensor device is a screen-printed carbon electrode (SPCE) or a membrane strip biosensor.

15. The kit of claim 13, wherein the first and second conjugating moieties are selected from the group consisting of biotin, avidin, and streptavidin.

16. The kit of claim 13, wherein:
(i) the further binding member is a glycan comprising a biotin moiety as the first conjugating member;
(ii) the biosensor comprises streptavidin as the second conjugating member bound to the zone on the surface, and
(iii) the glycan is immobilized on the surface of the biosensor by conjugation of the biotin moiety with the streptavidin moiety.

17. The kit of claim 16, further comprising gold nanoparticles (AuNP) at the surface to which the glycan is immobilized.

18. The kit of claim 6, wherein the binding pair member and the further binding pair member are capable of simultaneously or sequentially binding a virus strain or a virus surface protein, thereby forming a triplex comprising the binding pair member of the BEAM nanoparticle and the further binding pair member bound to the virus strain or said virus surface protein.

19. A biosensor device comprising a glycan immobilized on a detection surface of the biosensor device.

20. A triplex comprising:
(a) the biologically enhanced, electrically active magnetic (BEAM) nanoparticle composition of claim 1;
(b) a further binding pair member that specifically recognizes a subtype of the virus strain or the virus surface protein specifically recognized by the binding pair member of the BEAM nanoparticle composition; and
(c) a virus or virally derived material comprising a virus strain or a virus surface protein, or a mutant or fragment thereof, wherein the virus or virally derived material is bound to both the binding pair member of the BEAM nanoparticle composition and the further binding pair member.

21. A method for detecting the presence of a virus strain or a virus surface protein in a sample, the method comprising:
(a) providing the triplex of claim 20; and
(b) detecting the triplex.

22. The method of claim 21, wherein providing the triplex in part (a) comprises:
(i) immobilizing the further binding pair member on a surface;
(ii) contacting the further binding pair with the sample for a time sufficient to bind any virus or virally derived material present in the sample to the further binding pair member, thereby forming a viral-further binding pair member conjugate; and
(iii) contacting the viral-further binding pair member conjugate with the BEAM nanoparticle composition for a time sufficient to bind the binding pair member of the BEAM nanoparticle composition to the virus or virally derived material of the viral-further binding pair member conjugate, thereby forming the triplex immobilized on the surface.

23. The method of claim 21, wherein providing the triplex in part (a) comprises:
(i) contacting the further binding pair member and the BEAM nanoparticle composition with the sample for a time sufficient to bind any virus or virally derived material present in the sample to the further binding pair member and the binding pair member of the BEAM nanoparticle composition, thereby forming the triplex; and
(ii) immobilizing the triplex on a surface.

24. The method of claim 21, wherein providing the triplex in part (a) comprises:
(i) immobilizing the further binding pair member on a surface;
(ii) contacting the BEAM nanoparticle composition with the sample for a time sufficient to bind any virus or virally derived material present in the sample to the binding pair member of the BEAM nanoparticle composition, thereby forming a viral-BEAM nanoparticle conjugate; and
(iii) contacting the a viral-BEAM nanoparticle conjugate with the further binding pair member for a time sufficient to bind the further binding pair member to the viral-BEAM nanoparticle conjugate, thereby forming the triplex immobilized on the surface.

25. The method of claim 21, wherein providing the triplex in part (a) comprises:
(i) contacting the further binding pair with the sample for a time sufficient to bind any virus or virally derived material present in the sample to the further binding pair member, thereby forming a viral-further binding pair conjugate;
(ii) immobilizing the viral-further binding pair conjugate on a surface; and
(iii) contacting the viral-further binding pair conjugate with the BEAM nanoparticle composition for a time sufficient to bind the binding pair member of the BEAM nanoparticle composition to the virus or virally derived material of the viral-further binding pair conjugate, thereby forming the triplex immobilized on the surface.

26. The method of claim 21, further comprising magnetically separating the triplex or a magnetic component thereof from a liquid medium and concentrating the triplex or the magnetic component thereof prior to detecting the triplex in part (b).

27. The method of claim 21, wherein the sample is saliva or serum obtained from a mammal.

28. The method of claim 21, wherein the sample is saliva or serum obtained from a human.

29. The method of claim 21, wherein the virus surface protein, or a mutant or fragment thereof, is from a recombinant source.

30. The method of claim 21, wherein detecting the triplex comprises (i) acid-doping the conductive polymer of the triplex and then (ii) performing cyclic voltammetry to a biosensor device to which the triplex is immobilized to detect the acid-doped triplex.

31. The method of claim 21, wherein the virus or virally derived material comprising the virus strain or the virus surface protein, or a mutant or fragment thereof is prepared from the sample, or contained in the sample.

32. The method of claim 21, further comprising determining that the virus strain or the virus surface protein is present in the sample.
